SEQUENCE LISTING (13 sequences)

SEQ ID NO: 1 - DNA, 20 bp, Homo sapiens
gtgtgtaattatgtgcttag

SEQ ID NO: 2 - DNA, 20 bp, Homo sapiens
cttatttgcatatttccagt

SEQ ID NO: 3 - DNA, 20 bp, Homo sapiens
caggttgcctgtttgttttc

SEQ ID NO: 4 - protein, 50 aa, Homo sapiens
DPKTYRRSYTHAKPPYSYISLITMAIQQSPNKMLTLSEIYQWIMDLFPFY

SEQ ID NO: 5 - protein, 50 aa, Mus musculus; identical to SEQ ID NO: 4

SEQ ID NO: 6 - protein, 50 aa, Rattus norvegicus; identical to SEQ ID NO: 4

SEQ ID NO: 7 - protein, 49 aa, Gallus gallus
DPKTYRRSYTHAKPPYSYISLITMAIQQSPNKMLTLSEIYQWIMDLFPF

SEQ ID NO: 8 - protein, 50 aa, Xenopus laevis; identical to SEQ ID NO: 4

SEQ ID NO: 9 - protein, 50 aa, Drosophila melanogaster
DPKTYRRSYTHAKPPYSYISLITMAIQNNPTRMLTLSEIYQFIMDLFPFY

SEQ ID NO: 10 - protein, 50 aa, Homo sapiens
DAKTFKRSYPHAKPPYSYISLITMAIQRAPSKMLTLSEIYQWIMDLFPYY

SEQ ID NO: 11 - protein, 50 aa, Mus musculus
DAKTFKRSYPHAKPPYSYISLITMAIQQAPSKMLTLSEIYQWIMDLFPYY

SEQ ID NO: 12 - protein, 49 aa, Homo sapiens
MPKGYRAPAHAKPPYSYISLITMAIQQAPGKMLTLSEIYQWIMDLFPYY

SEQ ID NO: 13 - protein, 50 aa, Mus musculus
MAKGYRRPLAHAKPPYSYISLITMAIQQAPGKMLTLSEIYQWIMDLFPYY

(19) United States
(12) Patent Application Publication
(10) Pub. No.: US 2007/0166710 A1
(43) Pub. Date: Jul. 19, 2007

(54) Methods for inhibiting adipogenesis and for treating type 2 diabetes

(76) Inventors: Markus Stoffel, New York, NY (US); Christian Wolfrum, New York, NY (US); David Q. Shih, New York, NY (US); Satoru Kuwajima, New York, NY (US); Daniel Besser, New York, NY (US)

Correspondence Address: HOFFMANN & BARON, LLP, 6900 JERICHO TURNPIKE, SYOSSET, NY, 11791, US

(21) Appl. No.: 10/551,392

(22) PCT Filed: Mar. 31, 2004
(86) PCT No.: PCT/US04/09954

§ 371 Date: Sep. 5, 2006

Related U.S. Application Data
(60) Provisional application No. 60/459,010, filed on Mar. 31, 2003.
(60) Provisional application No. 60/459,011, filed on Mar. 31, 2003.

(51) Int. Cl. C12Q 1/68; G01N 33/53; A61K 48/00; A61K 38/17

(52) U.S. Cl. 435/6; 435/7.1; 514/12; 514/44

(57) ABSTRACT

The present invention relates to methods for inhibiting adieogenesis and methods for treating obesity, metabolic syndrome and non-insulin dependent diabetes mellitus by administering an agent that increases Foxa-2 or Fxr, or an agent that activates Fxr. The invention is further related to methods for identifying agents that increase Foxa-2 or Fxr, or activate Fxr, and the use of such agents for treatment of obesity, metabolic syndrome and non-insulin dependent diabetes mellitus. Methods of identifying agents that mediate the phosphorylation of the transcription factor Foxa-2 are provided. Such agents are useful in methods of treating Type 2 diabetes.

### CROSS-REFERENCE TO RELATED APPLICATIONS

This application claims the benefit of U.S. Provisional Application No. 60/459,010 filed Mar. 31, 2003 and U.S. Provisional Application No. 60/459,011 filed Mar. 31, 2003, the disclosures of which are incorporated herein by reference.

### STATEMENT REGARDING FEDERALLY FUNDED RESEARCH

This invention was made with government support under National Institutes of Health Grant RO1 DK55033-04 and Medical Scientists Training Program Grant GM07739. The government may have certain rights in the invention.

### BACKGROUND OF THE INVENTION

Obesity results from the expansion of white adipose tissue (WAT) by the recruitment of adipocyte precursor cells, and is a major cause of insulin resistance and diabetes. The process of adipocyte differentiation is the focus of extensive research, and a cascade of transcription factors that are responsible for this conversion have been identified. Rosen et al. (2000) Annv. Rev. Cell. Dev. Biol. 16:145-171. In addition, a number of factors that inhibit adipogenesis have been identified including the extracellular signaling molecules interleukin-1, tumor necrosis factor α (TNFα) and the cell surface protein preadipocyte factor-1 (Pref-1). Ohsumi et al. (1994) Endocrinol. 135:2279-2282; Smas et al. (1993) Cell 73:725-734.

The hepatocyte nuclear factor 3 (Hnf-3)/forkliead family of transcription factors in mammals includes three genes designated as Foxa-1 (Hnf-3α), Foxa-2 (Hnf-3β) and Foxa-3 (Hnf-3γ). Kaestner et al. (1994) Genomics 20: 377-385. These factors have in common a highly conserved 100 amino acid winged-helix motif that is responsible for monomeric recognition of specific DNA target sites. Brennan (1993) Cell 74: 773-776. Foxa proteins play a central role in maintaining normal metabolism by regulating gene expression of rate-limiting enzymes of gluconeogenesis and glycogenolysis in the liver and kidney, including phosphoenolpyruvate carboxykinase (Pepck) and glucose-6-phosphatase (G6pc), and by regulating glucagqn and Pdx-1 gene expression in pancreatic α- and β-cells, respectively. O'Brien et al. (1995) Mol. Cell Biol. 15: 1747-1758; Gerrish et al. (2000) J. Biol. Chem. 275: 3485-3492; Lee et al. (2002) Diabetes 51: 2546-2551; Shih et al. (1999) Proc. Natl. Acad. Sci. USA 96: 10152-10157; Tan et al. (2002) Hepatology 35: 30-39.

It has been surprisingly discovered in accordance with the present invention that Foxa-2 plays a crucial role in the regulation of adipocyte differentiation and metabolism.

The nuclear hormone receptor farnesoid X receptor (Fxr) is a bile acid-activated receptor that regulates hepatic biosynthesis of bile acids from cholesterol. Fxr positively regulates the expression of several genes involved in lipoprotein metabolism, and thus contributes to the maintenance of proper plasma cholesterol and triglyceride levels. In accordance with the present invention, it has been found that Fxr also plays an important role in adipocyte differentiation and metabolism.

In the liver insulin regulates gene expression of enzymes of gluconeogenesis and glycogenolysis by suppressing transcriptional activity. These pathways ensure that hepatic glucose production is suppressed in the fed state (when insulin levels are increased) and glucose levels are maintained in times of starvation (when serum insulin is low and glucagon is increased). Granner et al. (1983) Nature 305: 549-551. Normal integrative function of the liver in the regulation of lipid and glucose metabolism is impaired in type 1 and type 2 diabetes. Untreated type 1 diabetes leads to virtually absent plasma insulin levels and hyperglycemia due to increased hepatic production of glucose combined with diminished peripheral utilization. Ketoacidosis results from increased mobilization of fatty acids from adipose tissue combined with accelerated synthesis of 3-hydroxybutyrate and acetoacetate. Casteels et al. (2003) Rev. Endocr. Metab. Disord. 4:159-66. In contrast, hyperinsulinemia is one of the hallmarks of type 2 diabetes and predictable hyperanabolic effects of high circulating insulin levels include glycogen accumulation, high rates of fatty acid biosynthesis and fatty acid esterifications at the expense of a reduced capacity for fatty acid oxidation and an accelerated production of VLDL and hypertriglyceridemia. Lewis et al. (2002) Endocr. Rev. 23:201-229. However, the precise mechanisms by which insulin regulates these metabolic pathways are incompletely understood.

Stimulation of the insulin receptor results in the activation of two major pathways: 1) the nitrogen-activated protein (MAP) kinase cascade and 2) the phosphatidylinositol 3-kinase (PI 3-kinase) pathway. The serine/threonine kinase PKB/Akt is one downstream target of phosphatidylinositol 3-kinase (PI3-kinase) and plays an important role in mediating effects of insulin on hepatic carbohydrate, lipid and protein metabolism. Franke et al. (1995) Cell 81: 727-736; Franke et al. (1997) Science 275: 665-668; Hardt et al. (2002) Circ. Res. 90: 1055-1063. Upon activation, Akt is translocated to the nucleus where it exerts effects on gene activity by phosphorylation of target proteins like Gsk3, Bad and Fkhrl1. Meier et al. (1999) J. Recept. Signal Transduct. Res. 19: 121-128; Datta et al. (1999) Genes Dev. 13: 2905-2927. Genetic studies of the PI3-kinase-Akt signaling pathway in the nematode C. elegans have established that this signaling cascade suppresses the function of the transcription factor daf16, which belongs to the forkhead/winged-helix family of transcription factors. Mutations in the Insulin/Igf-1 receptor homologue (daf-2), the catalytic subunit of PI3-kinase (agε-1), or Akt (akt1 and akt2) result in increased longevity and constitutive cauei fonnation, a stage of developmental arrest and reduced metabolic activity that enhances survival periods of food deprivation and other environmental stresses. Kenyon et al. (1993) Nature 366: 461-464. In each case, mutation of daf-16 restored normal life span and prevented entry into dauer stage. Gottlieb et al. (1994) Genetics 137: 107-120; Ogg et al. (1997) Nature 389:

994-999. Subsequently, studies in mammals have shown that the Fkhr (Foxo-1), Fkhrl1 (Foxo-3) and AFX (Foxo-4) genes, members of the human forkhead family, also constitute downstream targets of Akt. Biggs et al. (1999) Proc. Natl. Acad. Sci USA 96: 7421-7426; Brunet et al. (1999) Cell 96: 857-868; Kops et al. (1999) Nature 398: 630-634. For instance, the Foxo-1 protein has been shown to be phosphorylated by Akt, which causes repression of transcriptional activity of insulin growth factor binding protein-1 (Igfbp-1), and G6pc. Nakae et al. (2001) J. Clin. Invest. 108: 1359-1367. Furthermore, genetic studies in mice have provided evidence that downstream components of the insulin/Igf-1 signaling pathway are essential for normial energy homeostasis and growth. Mice lacking Akt2 have an impaired ability of insulin to inhibit glucose production in the liver and muscle. Cho et al. (2001) Science 292: 1728-1731. In contrast, mice lacking Akt1 have normal glucose homeostasis, but impaired fetal and postnatal growth. Cho et al. (2001) J. Biol. Chem. 276: 38349-38352.

In accordance with the present invention, it has been discovered that activation of PI3-kinase-Akt by insulin induces the phosphorylation of Foxa-2, leading to nuclear exclusion and inhibition of Foxa-2 dependent transcriptional activity.

### BRIEF SUMMARY OF THE INVENTION

The present invention provides a method for inhibiting adipogenesis comprising contacting a cell with an agent that increases levels of Foxa-2 mRNA and/or protein.

The present invention further provides a method for inhibiting adipogenesis comprising contacting a cell with an agent that increases the levels of Fxr mRNA and/or protein, or an agent that activates Fxr.

The present invention further provides a method of treating obesity, metabolic syndrome and/or Type 2 diabetes (non-insulin dependent diabetes mellitus) comprising administering to a subject in need of such treatment a composition comprising an agent that increases Foxa-2.

The present invention further provides a method of treating obesity, metabolic syndrome and/or non-insulin dependent diabetes mellitus comprising administering to a subject in need of such treatment a composition comprising an agent that increases the levels of Fxr mRNA and/or protein, or an agent that activates Fxr.

In another embodiment, the present invention provides methods of identifying agents that increase Foxa-2, agents that activate Fxr and agents that increase Fxr. Such agents are useful for the treatment of obesity, metabolic syndrome, and Type 2 diabetes.

Agents that increase Foxa-2, agents that activate Fxr and agents that increase Fxr and compositions comprising such agents are also provided by the present invention.

The present invention further provides a method of identifying agents that prevent nuclear exclusion of Foxa-2 in hepatocytes. Agents that prevent nuclear exclusion and compositions comprising such agents are also provided by the present invention. Such agents are useful for the treatment of obesity type 2 diabetes and hyperinsulinemia.

The present invention also provides a method of identifying agents that mediate the phosphorylation of Foxa-2. Such agents are useful for the treatment of odesity, type 2 diabetes and hyperinsulinemia.

In another embodiment, the present invention provides methods of treating Type 2 diabetes and hyperinsulinemia comprising administering to a subject in need of such treatment a composition comprising an agent that inhibits the phosphorylation of Foxa-2.

Agents that mediate the phosphorylation of Foxa-2 and compositions comprising such agents are also provided by the present invention.

### BRIEF DESCRIPTION OF THE DRAWINGS

FIGS. 1a-f demonstrate the expression of Foxa-2 in adipose tissue. FIG. 1a is a Western blot of liver and adipose tissue extracts analyzed for Foxa-2 expression. FIG. 1b is a Northern blot of visceral and subcutaneous fat from wt and ob/ob mice analyzed for Foxa-2 expression. FIG. 1e is a Western blot of preadipocyte (Pre) and adipocyte (Ad) protein extracts. FIGS. 1d-f are images from confocal image inimunostaining of visceral fat from an ob/ob mouse.

FIGS. 2a-c demonstrate that Foxa-2 expression is induced by insulin. FIG. 2a is a graph depicting the correlation of Foxa-2 and Foxa-2 mRNA expression with plasma insulin concentration in various mouse models. FIGS. 2b and 2c demonstrate the effect of insulin and other factors on Foxa-2 expression in primary adipocytes from wt (FIG. 2b) and ob/ob (FIG. 2c) mice.

FIGS. 3a-e demonstrate that Foxa-2 inhibits adipocyte differentiation in 3T3-L1 cells.

FIGS. 4a-e show that Foxa-2 regulates genes involved in glucose uptake, glycolysis, lipolysis and energy dissipation.

FIGS. 5a-n depict the development of diet-induced obesity and metabolic analysis of primary adipocytes of Foxa-2^{+/-} and wildtype littermates.

FIG. 6 demonstrates the expression of Fxr in embryoid bodies deficient for Foxa-2.

FIG. 7 is a graph depicting transactivation of murine and human Fxr by members of the hepatocyte nuclear factor (HNF) family.

FIG. 8 depicts an electrophoretic mobility shift analysis of the Foxa-binding site in the Fxr-1 promoter.

FIGS. 9a and b depict the de novo expression of Fxr-1 in adipose tissue of ob/ob mice, and primary adipocytes of lean mice stimulated with insulin.

FIGS. 10a-d depict the effect of insulin on Foxa-2 activity. FIG. 10a is a bar graph depicting relative Foxa-2 activity in HepG2 cells transfected with an expression vector for Foxa-2 and pPepck-Luc (gray bars) or p6xCdx-TkLuc (black bars). Cells were treated with insulin alone or in the presence of Ly294002 or PD98059. FIG. 10b is a bar graph depicting relative Foxa-2 activity in HepG2 cells transfected with p6xCdx-TkLuc and treated with insulin alone or in the presence of Ly294002 or PD98059. FIG. 10c shows an RT-PCR analysis of Foxa-1-3 and target genes in primary hepatocytes grown in the presence of insulin (50 nM), Ly294002 and PD98059 for 6 hours prior to gene expression analysis. FIG. 10d is a bar graph depicting relative Foxa-2 activity in HepG2 cells transfected with an expression vector for Foxa-1, Foxa-2, Akt or Akt_{K179A} alone or in combination, using p6xCdx-TkLuc as reporter gene. In all experiments luciferase activity was normalized to β-Gal activity. Values are mean of 6 independent experiments±SD.

FIG. 11a is a sequence alignment of orthologous and paralogous members of the Foxa family. FIG. 11b is a bar graph depicting relative Foxa-2 activity in HepG2 cells transfected with expression vectors for Foxa-2, Foxa-2_{T156A} or Foxa-2_{R153K} together with Akt in varying concentrations. p6xCdx-TkLuc was used as the reporter gene. FIG. 11c is a bar graph depicting relative Foxa-2 activity in HepG2 cells transfected with expression vectors for Foxa-2, Foxa-2_{T156A} or Foxa-2_{R153K} together with Akt at the indicated concentrations. pPepck-Luc was used as the reporter gene. In all experiments luciferase activity was normalized to β-Gal activity. Values are mean of 6 independent experiments.

FIG. 12 is a Western blot of cell lysates (upper panel) and precipitates (lower panel) of HEK/293 cells transfected with expression vectors for Foxa-2, Foxa-2_{T156A} or Foxa-2_{R153K} together with HA-Akt. HA-Akt was precipitated using an HA-antibody; Foxa-2 was precipitated using an anti-Foxa-2 antibody. Cell lysates and precipitates were separated by SDS-PAGE and analyzed for Foxa-2 or Akt by Western blotting.

FIG. 13 is an autoradiograph demonstrating that Akt can phosphorylate Foxa-2 on residue TI56. Recombinant GST-Foxa-2, GST-Foxa-22_{T156A} or a positive control were incubated with precipitated HA-Akt or HA-LCK-Akt and [γ^{32P}]-ATP. Proteins were separated by SDA-PAGE and phosphorylated proteins were visualized by autoradiography.

FIG. 14a depicts the results of an electrophoretic mobility shift assay of cell extracts (CE) from insulin-stimulated HEK/293 cells transfected with Foxa-2 or Foxa-2_{T156A} together with Akt. The Foxa-2 binding site of Igfbp-1 was used to shift proteins; a consensus Foxa-2 binding site was used for competition. Supershift was performed using anti Foxa-2 antibody. FIG. 14b depicts untransfected and Akt-transfected HepG2 cells treated with insulin (50 nM), Ly294002 or PD98059 (10 μm), alone or in combination, decorated with anti-Foxa-1 or Foxa-2 antibodies, and visualized with an anti-rabbit IgG-Alexa 480 antibody using laser scanning microscopy. FIG. 14c depicts HepG2 cells transfected with expression vectors for either HA-Foxa-2 or HA-Foxa-2_{T156A} and treated with insulin (50 nM), decorated with an anti-HA antibody, and visualized with an anti-rabbit IgG-Alexa 480 antibody using laser scanning microscopy. Control cells were starved for 10 hours. All other experiments were performed in medium containing 10% fetal calf serum.

FIG. 15a is an immunoblot of Foxa-2 and Foxo-1 in liver nuclear extracts of fed (n=3) and starved (n=3) mice. Anti-TATA binding protein (Tbp) antibodies were used as a loading control. FIGS. 15b and 15c are immunoblots of Foxa-2 and Foxo-1 in nuclear extracts of livers from mice perfused with different concentrations of insulin. FIG. 15d is a Western blot of Foxa-2 immunoprecipitated with anti-Foxa-2 antibodies from whole cell extracts of livers obtained from fasted mice 30 minutes after injection of 10 ng insulin or PBS in the portal vein. Samples (n=3 for each group) were analyzed by immunoblot analysis with an antibody directed against phospho-TI56 Foxa-2 (top panel) or with an antibody directed against total Foxa-2 protein (bottom panel).

FIG. 16 is an immunoblot of Foxa-2 from nuclear extracts of livers from mice that were infected with Ad-GFP or Ad-TI56A after a six hour fast. Nuclear extracts of mice were analyzed one day or two weeks after injection of recombinant adenovirus into the tied vein.

FIG. 17 is an electrophoretic mobility shift assay (EMSA) of Foxa-2 in nuclear extracts of livers of fasted wild type and Srebp-1c mice that were infected with Ad-GFP, Ad-Foxa-2 and Ad-TI56A.

FIG. 18 is a graph depicting glucose output of livers from ob/ob mice that were infected with Ad-GFP or Ad-TI56A one week prior to the study. Livers were perfused through the portal vein with a modified Krebs-Henseleit buffer and indicated insulin concentrations. Glucose concentrations in the effluent were collected and assayed using the glucose oxidase method. Measurements are means of n=3.

FIG. 19 is a Western blot depicting an analysis of phosphorylated Akt, total Akt and Irs-2 expression. Whole cell liver lylsates were prepared from perfused livers of Ad-GFP and Ad-TI56A infected mice at 50, 100 and 130 minute time points. Protein (20 μg) was separated by SDS-polyacrylamide gel electrophoresis (SDS-PAGE) and transferred to a nitrocellulose membrane. Membranes were probed with an anti-phosphopeptide Akt, total anti-Akt, anti-Irs-2, and anti-TATA binding protein (anti-TBP) antibodies as loading control.

FIG. 20a-f are graphs showing decreased β-oxidation and ketogenesis, increased plasma free fatty acid and triglyceride levels and reduced hepatic insulin sensitivity in Foxa-2^{+/-} mice compared to wildtype littermates. FIGS. 20a and b show the production of ¹⁴CO₂ as a measure for β-oxidation (a) and ketone body generation (b) from [1-¹⁴C] palmitic acid by mitochondria from livers of wildtype (Wt) or Foxa-2^{+/-} mice that were fed a normal (chow) or high fat (HF) diet are shown. FIGS. 20c and d show plasma triglyceride (c), and plasma free fatty acid (FFA) (d) concentrations of wildtype or Foxa-2^{+/-} mice that were fed a normal (chow) or high fat (HF) diet. * denotes P=0.05. FIGS. 20e and f show glucose output measurements from perfused livers of Foxa-2^{+/-} or wildtype (Wt) mice that were fed a chow diet (e) or a high fat (HF) diet (f) with buffer containing high (20 ng/ml) and low (0.5 ng/ml) concentrations of insulin. Values are means of n=3.

### DETAILED DESCRIPTION OF THE INVENTION

In accordance with the present invention it has been discovered that the winged forkhead transcription factor Foxa-2 (previously designated hepatocyte nuclear factor-3β, HNF-3β) is induced de novo in visceral and subcutaneous fat of genetic and diet-induced mammalian models of obesity. Foxa-2 expression can be induced by insulin in primary adipocytes, and Foxa-2 levels in fat positively correlate with fasting serum insulin concentrations of hyperinsulinemic animals. The expression of Foxa-2 inhibits adipocyte differentiation in vitro and activates genes involved in glucose and fat metabolism. Diet-induced obese mice with haplosufficiency in Foxa-2 develop increased adiposity compared to wildtype littermates, and adipocytes of these mice exhibit defects in glucose uptake and metabolism. These discoveries show that Foxa-2 is an insulin-regulated gene that inhibits adipocyte differentiation and plays a crucial role as a physiological regulator of adipocyte differentiation and metabolism. Induction of Foxa-2 expression stimulates a protective mechanism that counteracts excessive actions of insulin in preadipocytes and enhances insultin sensitivity in mature adipocytes.

Accordingly, the present invention provides a method for inhibiting adipogenesis comprising contacting a cell capable of adipogenesis with an agent that increases levels of Foxa-2 rnRNA and/or protein. In a preferred embodiment the agent induces expression of Foxa-2. The invention further provides a method of treating obesity, metabolic syndrome and/or non-insulin dependent diabetes mellitus comprising administering to a subject in need of such treatment a composition comprising an agent that increase Foxa-2. The term “Foxa-2” as used herein refers to Foxa-2 from any species. In a preferred embodiment Foxa-2 is mammalian Foxa-2. In a more preferred embodiment Foxa-2 is human Foxa-2.

Agents that induce expression of Foxa-2 can be identified by a screening method which provides another embodiment of a present invention. The method of screening for agents that induce Foxa-2 expression comprises contacting a plurality of cells that contain a Foxa-2 promoter operably linked to a coding sequence for Foxa-2 with a candidate agent, assaying for Foxa-2 expression in the presence and absence of the candidate agent, and comparing Foxa-2 expression in the presence and absence of the candidate agent, whereby an increase in Foxa-2 expression in the presence of the candidate agent is indicative of the identification of an agent that increases Foxa-2 expression.

In a preferred embodiment of the present method, the cells are mammalian cells. More preferably the cells are human. The cells may be cells that comprise the Foxa-2 gene but do not express Foxa-2 under normal culture conditions. Such cells include preadipocytes and adipocytes. In a preferred embodiment the cells are 3T3-L1 cells or primary preadipocytes or adipocytes of lean subjects. The cells may be isolated and cultured by conventional methods, or obtained commercially. Human preadipocytes and adipocytes are commercially available.

The cells may also be cells that have been engineered to contain a construct comprising the Foxa-2 promoter operably linked to the coding sequence for Foxa-2. Mammalian Foxa-2 genes are known in the art, and the promoters and coding regions have been sequenced and characterized. See, e.g. Kaestner (2000) TEM 11: 281-283. Different isoforms of Foxa-2 exist, and are derived from alternative first exons and differential splicing at the 5′ end of the gene. Sasaki et al. (1994) Cell 76: 103-115. It has been determined by 5′-RACE analysis that the adipoccyte-specific Foxa-2 isoform is encoded by the L1 transcript. In a preferred embodiment of the present invention, the coding sequence encodes the adipocyte-specific Foxa-2 isoform. In a preferred embodiment the Foxa-2 promoter for adipocyte expression is located upstream of exon L1. The mouse Foxa-2 promoter is known in the art and disclosed, e.g. at NCBI Genome database entry L25669 and by Sasaki et al. (1994) Cell 76: 103-115. The human Foxa-2 promoter is known in the art and disclosed, e.g. at NCBI accession number AL121722. Those of ordinary skill in the art can identify the promoter, as well as fragments, modifications and variants thereof that are effective to direct expression of Foxa-2 in adipocytes. Foxa-2 coding regions are also known in the art. In a preferred embodiment, the Foxa-2 coding region is the mouse sequence disclosed at NCBI Genome database entry U04197 or the human sequence disclosed at NCBI entry NM153675 and Yamada et al. (2000) Diabetologia 43: 121-124. Those of ordinary skill in the art can identify fragments, variants and modifications of these sequences that retain the ability to encode a Foxa-2 polypeptide having the function of inhibiting adipocyte differentiation and increasing insulin sensitivity in adipocytes. The term “operably linked” is understood to mean that the promoter directs the expression of protein encoded by the coding sequence.

The construct can be introduced into a host cell by methods known in the art. The construct is preferably provided within an expression vector that is suitable for introduction into a host cell, and that contains nucleic acid sequences that control expression. Expression vectors are well-known in the art, and may be constructed by conventional methods. A starting vector may be obtained commercially and modified to include the present construct. In a preferred embodiment, the vector is the pGL2-Enhancer Vector (Promega).

The vector may be introduced into a host cell by methods well-known in the art. Transformation of a host cell may be accomplished, for example, by transfection, infection, electroporation, microinjection, and other well-known techniques set forth in laboratory manuals including Sambrook et al. (2001) Molecular Cloning: A Laboratory Manual, 3^{rd} ed., Cold Spring Harbor Laboratory Press, Cold Spring Harbor, N.Y., incorporated herein by reference. Host cell lines are well-known in the art and are commercially available. Cell lines stably transformed with the vector of the invention are preferred. In a preferred embodiment, the host cell is a mammalian preadipocyte or adipocyte. More preferably, the host cell is a human preadipocyte or adipocyte.

Host cells comprising the Foxa-2 promoter and coding sequence are cultured under standard conditions known in the art and contacted with a candidate agent.

Candidate agents include any chemical compound, and may be naturally occurring or synthetic. Combinatorial libraries of candidate agents may be used. In a preferred embodiment, well-known automated methods of high throughput screening are used to assay candidate agents. Agents that can be transported into adipocytes or formulated for transport into adipocytes are preferred.

Foxa-2 expression may be assayed by detecting Foxa-2 mRNA by conventional methods, for example by Northern blotting using Foxa-2 specific probes or quantitative polymerase chain reaction (PCR) using Foxa-2 specific primers. Foxa-2 expression may also be assayed by detecting Foxa-2 protein, for example by Western blotting or immunohistochemistry using anti-Foxa-2 antibodies. Such antibodies may be generated by methods known in the art or obtained commercially.

An increase in Foxa-2 expression in the presence of the candidate agent relative to expression in the absence of the agent is defined herein as an increase that is detectable by any of the foregoing methods. Agents identified by the screening method of the invention may be used as potential therapeutics or may serve as lead compounds for the development of therapeutics.

The present invention also provides a method of screening for compounds that induce Foxa-2 expression comprising contacting a plurality of cells that contain a Foxa-2 promoter operably linked to the coding sequence of a reporter gene with a candidate agent, assaying for the expression of the reporter in the presence and absence of the candidate agent, and comparing expression of the reporter in the presence and absence of the candidate agent, whereby an increase in the expression of the reporter in the presence of the candidate agent is indicative of an agent that increases Foxa-2 expression.

The method is performed as described hereinabove except that the Foxa-2 coding sequence is replaced by a reporter sequence, and detection of expression of Foxa-2 is replaced by detection of expression of the reporter.

Reporter genes that encode easily assayable reporter proteins are well-known in the art. In general, a reporter gene is a gene which is not normally present or expressed in the host cell, and which expresses a protein having an easily detectable property. Preferred reporter genes include the chloramphenicol acetyl transferase (cat) gene, the beta-galactosidase (gal) gene, the beta-glucuronidase (gus) gene, the green fluorescence protein (GFP) gene, and the luciferase (luc) gene. The methods of detection of these reporters are well-known in the art, and are dictated by the nature of the reporter. For example, beta-galactosidase hydrolyzes galactosides to yield detectable colored products.

In a preferred embodiment of these methods, the host cell is a 3T3-L1 cell that has been stably transformed with a construct comprising the Foxa-2 promoter operably linked to the coding sequence of the luciferase gene.

Agents identified by the foregoing screening methods are useful for inhibiting adipogenesis and for treating obesity, metabolic syndrome, and diabetes. The present invention provides compositions comprising such agents. The compositions may further comprise a diluent, carrier, solubilizer, emulsifier, preservative and/or adjuvant, and are preferably formulated for transport into adipocytes.

The formulation of phannaceutical compositions is generally known in the art and reference can conveniently be made to Remington's Pharmaceutical Sciences, 18th ed., Mack Publishing Co., Easton, Pa. Formulations for use in present invention must be stable under the conditions of manufacture and storage and must also be preserved against the contaminating action of microorganisms such as bacteria and fungi. Prevention against microorganism contamination can be achieved through the addition of various antibacterial and antifungal agents.

The pharmaceutical forms of the present agents suitable for administration include sterile aqueous solutions or dispersions and sterile powders for the extemporaneous preparation of sterile injectable solutions or dispersions. In all cases, the form must be sterile and must be fluid to the extent that easy syringability exists. Typical carriers include a solvent or dispersion medium containing, for example, water buffered aqueous solutions (i.e., biocompatible buffers), ethanol, polyols such as glycerol, propylene glycol, polyethylene glycol, suitable mixtures thereof, surfactants, or vegetable oils.

Sterilization can be accomplished by any art-recognized technique, including but not limited to filtration or addition of antibacterial or antifungal agents, for example, paraben, chlorobutanol, phenol, sorbic acid or thimerosal. Further, isotonic agents such as sugars or sodium chloride may be incorporated in the subject compositions.

Production of sterile injectable solutions containing the subject agents is accomplished by incorporating these compounds in the required amount in the appropriate solvent with various ingredients enumerated above, as required, followed by sterilization, preferably filter sterilization. To obtain a sterile powder, the above solutions are vacuum-dried or freeze-dried as necessary.

The subject agents are thus compounded for convenient and effective administration in pharmaceutically effective amounts with a suitable pharmaceutically acceptable carrier and/or diluent in a therapeutically effective dose.

The methods of inhibiting adipogenesis and enhancing insulin sensitivity may also be accomplished by contacting a cell with Foxa-2 protein or a vector capable of expressing Foxa-2 protein in preadipocytes and/or adiopocytes. The vector may comprise a construct having a constitutive promoter operably linked to a nucleic acid encoding Foxa-2. The method of treating obesity, metabolic syndrome or diabetes may be accomplished by administering a composition comprising Foxa-2 protein or a vector capable of expressing Foxa-2 protein.

It has further been discovered in accordance with the present invention that adipogenesis may be inhibited by increasing or activating the nuclear hormone receptor Fxr. In particular, it has been discovered that Fxr is expressed de novo in adipocytes of obese (hyperinsulinemic) mice, and that Fxr expression can be induced by culturing primary adipocytes of lean mice in the presence of insulin. Increased levels of Fxr, or activation of Fxr by endogenous or synthetic ligands, is likely to lead to induction of genes that enhance insulin sensitivity in adipocytes. The term “Fxr” as used herein refers to Fxr from any species. In a preferred embodiment, Fxr is mammalian Fxr. In a more preferred embodiment, Fxr is human Fxr.

Accordingly, the present invention provides a method for inhibiting adipogenesis comprising contacting a cell capable of adipogenesis with an agent that increases levels of Fxr mRNA and/or protein, or an agent that activates Fxr. The invention further provides a method of treating obesity, metabolic syndrome and/or non-insulin dependent diabetes mellitus comprising administering to a subject in need of such treatment a composition comprising an agent that increases or activates Fxr.

A method of screening for agents that increase Fxr expression comprises contacting a plurality of cells that contain an Fxr promoter operably linked to a coding sequence for Fxr with a candidate agent, assaying for Fxr expression in the presence and absence of the candidate agent, and comparing Fxr expression in the presence and absence of the candidate agent, whereby an increase in Fxr expression in the presence of the candidate agent is indicative of the identification of an agent that increases Fxr expression.

In a preferred embodiment, the cells are mammnalian cells. More preferably, the cells are human. The cells may be cells that comprise the Fxr gene but do not express Fxr under normal culture conditions. Such cells include preadipocytes and adipocytes. In a preferred embodiment the cells are 3T3-L1 cells.

The cells may also be cells that have been engineered to contain a construct comprising the Fxr promoter operably linked to the coding sequence of Fxr. Mammalian Fxr genes are known in the art, and the promoters and coding sequences have been sequenced and characterized. See, e.g. Chiang (2002) Endocrine Reviews 23:443-463 and U.S. Patent Application Publication 2003/0003520A1. In a preferred embodiment, the Fxr promoter is contained within a 1245 base pair fragment upstream of the coding sequence (ATG) of the human Fxr gene (NCBI nucleotide database NT-009743) and the Fxr coding sequence is provided at NCBI nucleotide database NM-005123 and disclosed by Forman et al. (1995) Cell 81: 687-693.

The constructs may be introduced into host cells as described above. Candidate agents are defined and screening may be performed as described above.

Fxr expression may be assayed by detecting Fxr mRNA by conventional methods, for example by Northern blotting using Fxr-specific probes, or by quantitative PCR using Fxr-specific primers. Fxr expression may also be assayed by detecting Fxr protein, for example by Western blotting or immunohistochemistry using anti-Fxr antibodies. Anti-Fxr antibodies may be generated by conventional methods.

An increase in Fxr expression is defined as an increase that is detectable by any of the foregoing methods.

The method of detecting agents that increase Fxr expression may be modified as described above to substitute the coding sequence of a reporter gene for the Fxr coding sequence, and assaying for expression for the reporter.

In a preferred embodiment of this method, the host cell is a 3T3-L1 cell that has been stably transformed with a construct comprising the Fxr promoter operably linked to the luciferase gene.

The present invention further provides a method for screening for agents that activate Fxr in adipose tissue. The method comprises contacting a plurality of cells that contain Fxr with a candidate agent, assaying for activation of Fxr in the presence and absence of a candidate agent, and comparing Fxr activation in the presence and absence of the candidate agent, whereby an increase in activation in the presence of the agent is indicative of the identification of an agent that activates Fxr. Activation of Fxr may be assessed by measuring reporter gene activity in cells that are transfected with a vector containing the Fxr promoter upstream of a reporter gene, e.g. the luciferase gene. Activation of Fxr may also be measured by measuring the increased expression of known target genes of Fxr, such as the small heterodimer partner (Shp) gene. Known activators of Fxr include naturally occurring agents such as bile acids (chenodeoxycholic acid (CDCA) and cholic acid (CA)), farnesol, juvenile hormone III, all-trans-retinoic acid and synthetic compounds such as GW4064 (Glaxo Smith Kline).

Agents that increase or activate Fxr in adipose tissue are useful for inhibiting adipogenesis and for the treatment of obesity, metabolic syndrome, and diabetes. Agents identified by the foregoing methods may be used as potential therapeutics or may serve as lead compounds for the development of therapeutics.

The present invention further provides compositions comprising agents that increase or activate Fxr. Such compositions may contain additional components, and may be formulated and delivered as described hereinabove.

In accordance with the present invention, it has further been discovered that activation of the PI3-kinase-Akt pathway by insulin induces the phosphorylation of Foxa-2. Foxa-2 is phosphorylated at a single conserved site (TI56) that is absent in Foxa-1 and Foxa-3. Phosphorylation of Foxa-2 leads to nuclear exclusion, and inhibition of Foxa-2 dependent transcriptional activity of genes involved in fatty acid oxidation, ketogenesis and glycolysis. Agents that inhibit Foxa-2 phosphorylation or otherwise prevent nuclear exclusion of Foxa-2 are thus useful in the treatment of Type 2 diabetes and hyperinsulinemia. Accordingly, the present invention further provides a method for treatment of Type 2 diabetes, insulin resistance or hyperinsulinemia comprising administering to a subject in need of such treatment an agent that inhibits Foxa-2 phosphorylation or othervise prevents nuclear exclusion of Foxa-2 in hepatocytes of said subject.

In a further embodiment, the present invention provides a method of identifying agents that inhibit the phosphorylation of Foxa-2. Phosphorylation of Foxa-2 leads to nuclear exclusion and inhibition of target genes, including genes of fatty acid oxidation, ketogenesis and glycolysis. Accordingly, agents that inhibit phosphorylation of Foxa-2 are useful for the treatment of type 2 diabetes and hyperinsulinemia.

The method of identifying agents that inhibit the phosphorylation of Foxa-2 comprises combining a candidate agent with a polypeptide having Akt kinase activity and a substrate comprising the phosphorylation domain of Foxa-2, assaying for phosphorylation of the substrate in the presence and absence of the candidate agent, and comparing phosphorylation in the presence and absence of the candidate agent, whereby a decrease in phosphorylation of the substrate in the presence of the candidate agent is indicative of the identification of an agent that inhibits phosphorylation of Foxa-2.

The polypeptide having Akt kinase activity may be naturally occurring or synthetic Akt, or fragments or modifications thereof that maintain serine/threonine kinase activity. Mammalian Akt, also known as protein kinase B (PKB), is known in the art and includes isoforms such as Akt 1, Akt 2 and Akt 3. Akt orthologs have also been cloned from other species including D. melanogaster and C. elegans. Datta et al. (1999) Genes Dev. 13: 2905-2927.

The structure of Akt has been well-characterized and is reviewed by Datta et al. The protein contains a central kinase domain with specificity for serine or threonine residues in the substrate, an amino-terminal domain that mediates lipid-protein and/or protein-protein interactions, and a carboxy terminus that includes a hydrophobic and protein rich domain. The primary structure is conserved evolutionarily except for the carboxy-terminal tail. Accordingly, one of ordinary skill in the art can determine fragments and modifications of Akt that maintain activity and are useful in the present method.

The polypeptide having Akt kinase activity can be purified or synthesized by methods known in the art, or obtained commercially. In a preferred embodiment, the polypeptide used in the present invention is human Akt 1 or human Akt 2. Recombinant Akt 1 or Akt 2 is preferred.

The substrate comprising the phosphorylation domain of Foxa-2 is a peptide or polypeptide comprising a domain having the amino acid sequence RRSYTH. In a preferred embodiment the substrate is a Foxa-2 protein or a fragment or modification thereof comprising the phosphorylation domain. Mammalian Foxa-2 is known in the art. See e.g. Kaestner (2000) TEM 11: 281-283. In a preferred embodiment, the substrate is human Foxa-2. Human Foxa-2 may be purified or synthesized by methods known in the art.

The method may be performed by providing the polypeptide having Akt kinase activity and a substrate comprising the phosphorylation domain of Foxa-2 in a cell-free in vitro system under conditions for phosphorylation. The method may also be performed in a cell extract or cells into which nucleic acids encoding the polypeptide and substrate have been introduced, or in which each naturally occurs, or in which one naturally occurs and the other has been introduced by standard methods of recombinant technology.

In a preferred embodiment, the method is performed in an in vitro system, the polypeptide having Akt kinase activity is mammalian Akt and the substrate is mammalian Foxa-2. Akt and Foxa-2 are preferably recombinantly produced, and in another preferred embodiment are human Akt and human Foxa-2.

Candidate agents include any chemical compound or molecule, and may be naturally occurring or synthetic. Combinatorial libraries of candidate agents may be used. In a preferred embodiment, well-known automated methods of high throughput screening are used to assay candidate agents.

Phosphorylation of the substrate may be measured by kinase assays known in the art. For example, in a typical in vitro kinase assay, the kinase and substrate are incubated in the presence of radiolabeled ATP, e.g. [γ^{32P}]-ATP, in a suitable buffer, e.g. a buffer containing MgCl₂ and MnCl₂. The substrate is immunoprecipitated, separated by SDS-PAGE, transferred to a membrane, and autoradiographed. The appearance of detectable bands on the autoradiograph indicates that the substrate has been phosphorylated. Phosphorylation may also be detected indirectly and in the absence of radioactivity, for example by using antibodies specific for the phosphorylated domain.

In the present method, the assay is performed in the presence and absence of the candidate agent. A detectable decrease in phosphorylation in the presence of the agent is defined as any decrease that is detectable by standard methods of assaying for phosphorylation, such as the in vitro kinase assay described above. Although the difference need not be quantitated, in a preferred embodiment the difference is at least about 10%.

In another embodiment, the present invention provides a method of identifying agents that inhibit nuclear exclusion of Foxa-2 in hepatocytes. The method comprises contacting a hepatocyte, under conditions whereby Foxa-2 exhibits nuclear exclusion, with a candidate agent; determining intracellular location of Foxa-2 in the presence and absence of the candidate agent; and comparing the intracellular location of Foxa-2 in the presence and absence of the agent, whereby an increase in nuclear localization of Foxa-2 in the presence of the candidate agent is indicative of the identification of an agent that inhibits nuclear exclusion of Foxa-2 in hepatocytes.

The hepatocytes may be cultured cells, for example HepG2 cells. Conditions under which Foxa--2 exhibits nuclear exclusion include treatment of cells with insulin, or overexpression of Akt, for example by transfecting the cells with a vector that expresses Akt. Agents that inhibit nuclear exclusion of Foxa-2 include agents that prevent the exit of Foxa-2 from the nucleus in response to treatment with insulin. In another embodiment, the hepatocytes may be within the liver of a mammal such as a mouse. In such an in vitro system, conditions under which Fox-2 exhibits nuclear exclusion include a fed state, injection of insulin, e.g. into the portal vein of the mammal, and perfusion of the liver with insulin. Hepatocytes that are under conditions whereby Fox-2 exhibits nuclear exclusion are also obtainable from insulin resistant mouse models such as the leptin deficient ob/ob, the lipoatrophic aP2-Srebp-1C, and the high fat diet-induced obese C57/B6 mice. Nuclear exclusion is defined herein to mean that at least 50%, and preferably at least 65%, and more preferably at least 80% of Foxa-2 in a hepatocyte is present in the cytoplasm.

Determination of the intracellular location of Foxa-2 is understood herein to mean determining whether Foxa-2 is present in the nucleus or cytoplasm. This determination may be made by methods known in the art, including Western blotting of nuclear or cytoplasmic extracts and immunohistochemistry. Determination of expression of Foxa-2-activated genes is also indicative of nuclear localization of Foxa-2.

An increase in nuclear localization of Foxa-2 is any increase that is detectable by standard methods such as Western blotting and immunohistochemistry. Although the increase need not be quantitated, in a preferred embodiment the increase is at least about 10%.

Candidate agents include any chemical compound or molecule, and may be naturally occurring or synthetic. Combinatorial libraries of candidate agents may be used. In a preferred embodiment, well-known automated methods of high throughput screening are used to assay candidate agents.

Agents identified by the methods of the present invention are useful for the treatment of diseases that may be ameliorated by altering the transcriptional activity of Foxa-2. The present invention provides compositions comprising such agents. The compositions may further comprise a diluent, carrier, solubilizer, emulsifier, preservative and/or adjuvant.

The formulation of pharmaceutical compositions is generally known in the art and reference can conveniently be made to Remington's Pharmaceutical Sciences, 18^{th} ed., Mack Publishing Co., Easton, Pa. Formulations for use in present invention must be stable under the conditions of manufacture and storage and must also be preserved against the contaminating action of microorganisms such as bacteria and fungi. Prevention against microorganism contamination can be achieved through the addition of various antibacterial and antifungal agents.

The pharmaceutical forms of the present agents suitable for administration include sterile aqueous solutions or dispersions and sterile powders for the extemporaneous preparation of sterile injectable solutions or dispersions. In all cases, the form must be sterile and must be fluid to the extent that easy syringability exists. Typical carriers include a solvent or dispersion medium containing, for example, water buffered aqueous solutions (i.e., biocompatible buffers), ethanol, polyols such as glycerol, propylene glycol, polyethylene glycol, suitable mixtures thereof, surfactants, or vegetable oils.

Sterilization can be accomplished by any art-recognized technique, including but not limited to filtration or addition of antibacterial or antifungal agents, for example, paraben, chlorobutanol, phenol, sorbic acid or thimerosal. Further, isotonic agents such as sugars or sodium chloride may be incorporated in the subject compositions.

Production of sterile injectable solutions containing the subject agents is accomplished by incorporating these compounds in the required amount in the appropriate solvent with various ingredients enumerated above, as required, followed by sterilization, preferably filter sterilization. To obtain a sterile powder, the above solutions are vacuum-dried or freeze-dried as necessary.

The subject agents are thus compounded for convenient and effective adminimstration in pharmaceutically effective amounts with a suitable pharmaceutically acceptable carrier and/or diluent in a therapeutically effective dose.

All references cited herein are incorporated herein in their entirety.

The following nonlimiting examples serve to further illustrate the present invention.

### EXAMPLE 1

### Materials and Methods

The following materials and methods were used in Examples 2-8.

Plasmids

The hPREF-1 promoter (681 bp) was cloned from a human lambda Fix II™ library (Stratagene). The murine m-Pk (989 bp), [HK-2] (771 bp), Lpl (1011 bp), Hs (599 bp) and Ucp-2 (877 bp) promoters were amplified using site specific primers and genomic DNA as template and cloned into the pGL2-Enhancer vector (Promega). The sequences of all the clones were confirmed by dideoxynucleotide sequencing.

Animals and Metabolic Cages

All animal models were maintained in C57B1/6J background and maintained on a 12 hours light/dark cycle in a pathogen-free animal facility. Oxygen consumption, CO₂ and heat production and food and water intake were simultaneously determined for 4 mice per experiment in an Oxymax metabolic chamber system (Columbus Instruments, Columbus, Ohio). Individual mice (15 weeks) were placed in a chamber with an airflow of 0.6 L/min and one reading per mouse was taken at 4-minute intervals over 24 hours. Resting metabolic parameters were determined by integrating values at periods of no activity.

Electrophoretic Mobility Shift Assay

Nuclear extracts were prepared as described by Stuempfle et al. (1996) Biotechniques 21:48-50, with minor modifications. Visceral fat from wt and ob/ob animals was washed in pre-chilled phosphate buffered saline (PBS) supplemented with protease inhibitor cocktail (Roche) and homogenized in sucrose buffer (20 mM Hepes pH 7.9, 25 mMA KCl, 2 M, sucrose, 20% (v/v) glycerol, 1 mM EDTA, protease inhibitor cocktail) using a dounce homogenizer. The homogenized tissue was centrifuged over a sucrose buffer cushion (100,000 g, 40 mm), and the nuclei were resuspended in lysis buffer (20 mM Hepes pH 7.9, 420 mM NaCl, 1.5 mM MgCl₂, 0.2 mM EDTA, protease inhibitor cocktail, 25% (v/v) glycerol). After 30 minutes of incubation, nuclear extracts were centrifuged (45,000 g, 30 mm) and the supernatant was snap frozen in liquid nitrogen. Protein content was measured by BCA-test.

Nuclear extracts from ob/ob fat (20 μg) were incubated with ³²P-labeled double-stranded oligonucleotide probes with either the wt or a mutated putative Foxa binding site from the PREF-1-, Lpl- and Ucp2-promoter. The reaction was performed in a mixture containing Hepes buffer (20 mM, pH 7.9), KCl (40 mM), MgCl₂ (1 mM), EGTA (0.1 mM), DTT (0.5 mM), 4% Ficoll and poly(dIdC) at room temperature for 10 minutes. Competition analysis was performed by incubating the cellular extracts and the probe with the non-labeled oligonucleotide. Supershift analysis was carried out by incubating the nuclear extracts with either anti-Foxa-1 or anti-Foxa-2 antibody disclosed by Ruiz I. Altaba et al. (1993) Mech. Dev. 44:91-108. The reaction mixture was loaded on a 6% non-denaturing polyacrylamide gel in TBE buffer (0.023 M Tris-borate, 0.5 mM EDTA) and run at 4° C. Bands were visualized by autoradiography. The sequence of the binding sites are PREF-1: 5′-GTGTGTAATTATGTGCTTAG-3′ (SEQ ID NO:1), Lpl: 5′ CTTATTTGCATATTTCCAGT-3′ (SEQ IN NO:2), Ucp-2: 5′-CAGGTTGCCTGTTTGTTTC-3′ (SEQ ID NO:3).

Cell Culture

3T3-L1 cells were maintained in DMEM with 4.5 g/l glucose, 10% fetal calf serum, 2 mM glutamine, 1 mM pyruvate and penicillin/streptomycin (Life Technologies, Inc.) in a humidified incubator at 5% CO₂. Cells were subcultured at a split ratio of 1:4. Adipocyte differentiation was induced as described by treating with 1 μM dexamethasone (Sigma) and 0.5 mM MIX (Sigma) for 8 days in the presence or absence of insulin (5 nM) (3). Incorporation of lipids was visualized by staining with Oil Red 0 (Sigma).

Transactivation Assay

3T3-L1 cells were grown to 60-70% confluence and subsequently transfected with the reporter genes (0.5 μg), pCMV-β-Gal as internal reference (0.5 μg) and the expression vectors for Foxa-1 and Foxa-2 (0.5 μg) or pcDNA3 alone as control by use of the transfection reagent Fugene6 according to manufacturer's protocol (Roche). Cells were grown for an additional 48 hours after transfection. Luciferase activity was measured using the Luciferase Detection System following the manufacturer's protocol (Promega). Luciferase was normalized for transfection efficiency by the corresponding β-galactosidase activity as described by Alam et al. (1990) Anal. Biochem. 188:245-254.

Generation of the Stable Cell Lines

3T3-L1 cells were plated at a density of 20,000 cells/cm² and transfected with the expression vector pcDNA3-Foxa-1, -Foxa-2, -Pref-1, using Fugene6 (Roche) as transfection reagent. The transfected cells were selected in 350 μg/ml of G418 (Life Technologies, Inc.) and approximately tvo hundred G418 resistant clones were pooled and expanded in selection medium. Expression of the stably transfected gene was confirmed by RT-PCR.

Reverse Transcriptase-PCR

Total RNA was extracted from cells and EBs using Trizol following manufacturer's instructions (Life Technologies, Inc.). Contaminating genomic DNA was removed by treating with 5 u of RNase-free DNase-I (Roche Molecular Biochemicals)/10 μg of RNA. cDNA was synthesized using moloney leukemia virus reverse transcriptase with dNTPs and random hexamer primers (Stratagene). The cDNAs provided templates for polymerase chain reactions (PCRs) using specific primers at annealing temperatures ranging between 60 and 65° C. in the presence of dNTPs, [α⁻³²P]dCTP, and Taq DNA polymerase. PCR synthesis for each primer pair was quantified at 15, 20, 25, and 30 cycles in a test reaction to ensure that the quantitative PCR amplification was in the linear range.

Northern Blot Analysis

Specific DNA probes were generated using the Highprime DNA labeling kit following the manufacturer's instructions (Roche). Total RNA fat tissue was prepared using Trizol as described by the manufacturer (Life Technologies, Inc.) and separated (30 μg per lane) on a 1% agarose gel containing 5% formaldehyde. After blotting onto a positively charged nylon membrane (Schleicher&Schuell), the blot was hybridized at 42° C. with the respective probe using Hybrisol hybridization buffer (Intergen).

Western Blot Analysis

Cytosolic protein extracts were separated by SDS-PAGE (13.5%) and transferred onto a nitrocellulose membrane (Schleicher&Schuell) by electroblotting. A-FABP was detected with anti-human aP2-antiserum (1:500) (F. Spener, Muenster, Germany) and goat anti-rabbit IgGs conjugated to HRP (1:10,000) in TBS supplemented with 5% nonfat dry milk. Foxa-2 was detected with anti-Foxa-2 antiserum (28) (1:1000) and goat anti-rabbit IgG conjugated to HRP (1:10,000). All antibodies were dissolved in 5% milk in TBS with 0.5% Tween-20. The blots were washed three times for 15 minutes between incubations. Membranes were incubated with primary antibodies overnight at 4° C. Incubations containing the secondary antibody were performed at room temperature for 1 hour. For visualization, the Renaissance Chemiluminescence Substrate (NEN, MA) was used.

Adipocyte Isolation and Metabolic Studies

Primary adipocytes were isolated as described by Rodbell (1964) J. Biol. Chem. 239:375-380. For the assessment of lipogenesis, lipolysis and glucose metabolism of a 10% isolated fat cell suspension at 5 mM glucose was used. Glucose transport of isolated adipocytes was measured by incubation for 30 minutes with 3 μM U-[¹⁴C]glucose with or without insulin stimulation as described by Black et al. (1995) J. Cell. Biochem. 58:435-463. The reaction was stopped by spinning through dinonyl phtalathe oil and the radioactivity quantified by scintillation counting. Glucose incorporated into triglycerides, lactate and CO₂ was measured after 2 hours incubation with 3 μM U-[¹⁴C]glucose in the absence or presence of 100 nM insulin as described by Tozzo et al. (1995) Am. J. Physiol. 268:E956-E964. Fatty acid de novo synthesis was analyzed by saponification of total lipids as described by Shakir et al. (1978) J. Lipid Res. 19:433-442 and quantification of radioactive label into fatty acids. Incorporation into lipid glycerol was calculated by subtracting fatty acid radioactive label from total lipid radioactive label.

To quantify lipolysis, isolated adipocytes (200 μl of a 10% isolated fat cell suspension) were incubated in the presence of adenosine deaminase and 10 μM PIA (N6[R-(−)-1-methyl-2-phenyl]adenosine), with or without 100 μM isoproterenol to produce maximal increase of lipolysis for 30 minutes in the presence or absence of insulin. Glycerol content of the incubation medium was determined using a radiometric assay as described by Susulic et al. (1995) J. Biol. Chem. 270:29483-20402.

Statistical Analysis

Results are given as mean±SD. Statistical analyses were performned by using a Student's t-test, and the null hypothesis was rejected at the 0.05 level. Linear regression was calculated using Origin (Microcal).

### EXAMPLE 2

### Expression of Foxa-2 (Hnf-3β) in Adipose Tissue

To identify genes that play a role in adipocyte differentiation and obesity, gene expression in adipose tissue of wildtype (wt) and obese (ob/ob) mice and monogenic (db/db) and polygenic (NZO) animal models of obesity was compared. Liver cell extracts from wt mice and adipocyte extracts from wt, ob/ob, db/db and NZO mice were separated by SDS-PAGE and analyzed by Western blotting for Foxa-2 expression. TATA binding protein (Tbp) expression was measured as a control for loading.

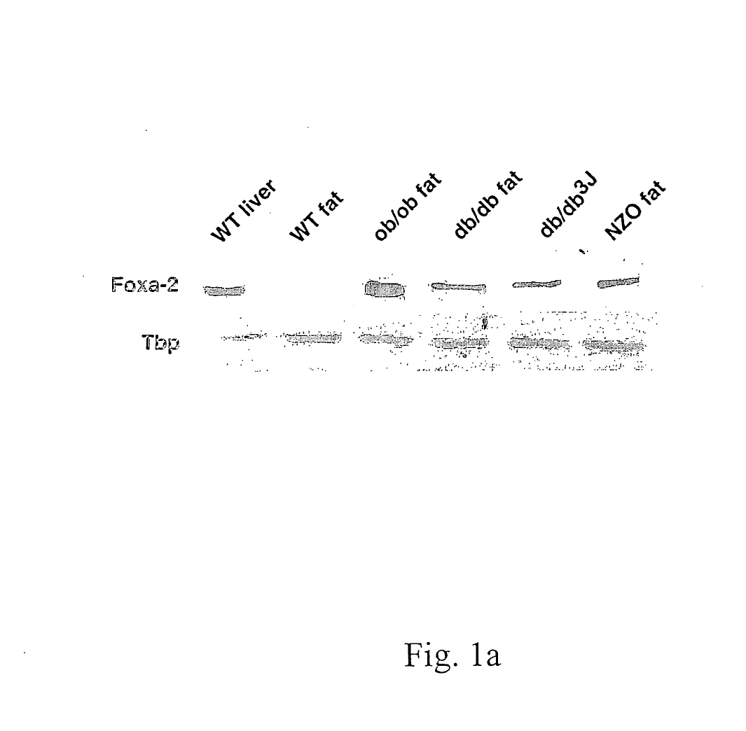

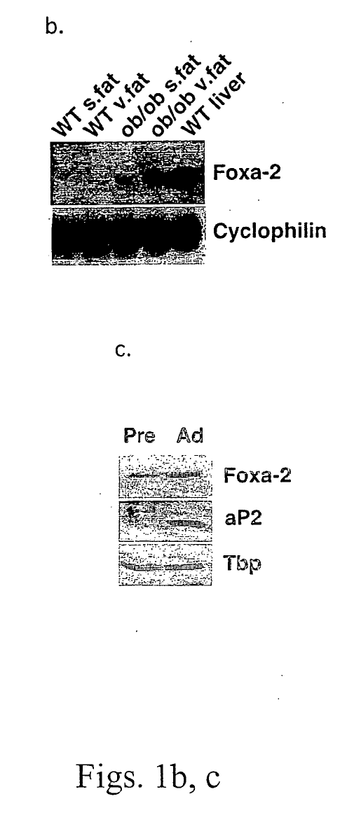

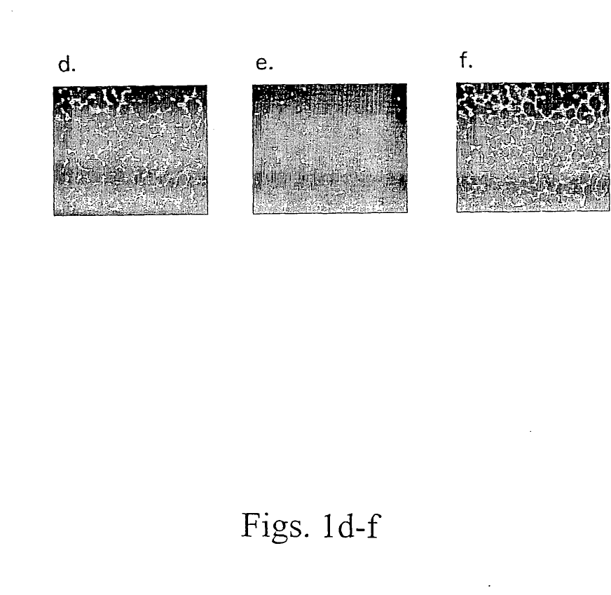

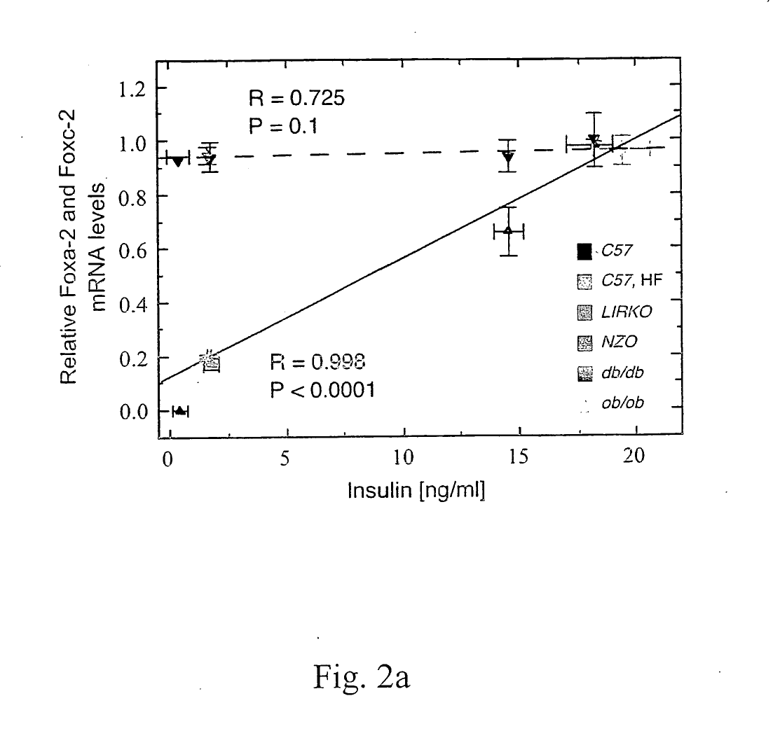

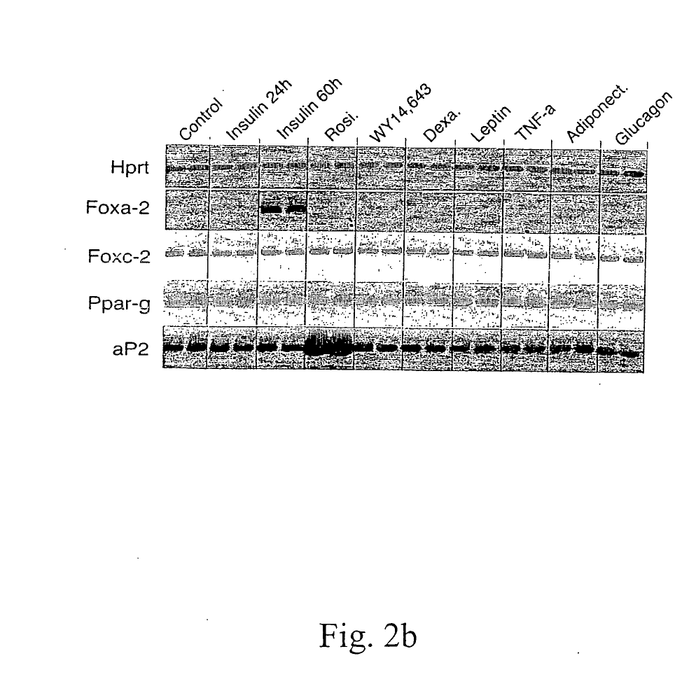

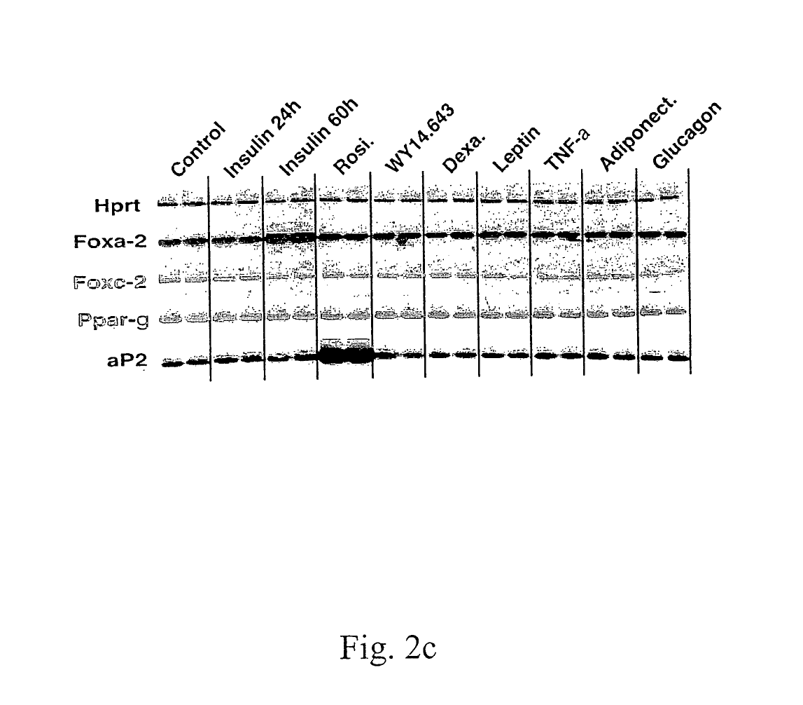

As shown in FIG. 1a, the forkhead transcription factor Foxa-2 (Hnf-3β) was undetectable in fat tissue of wt mice, and was expressed in adipose tissue of obese mice. Expression of Foxa-2 was also found in the monogenic (db/db) and polygenic (NZO) models of obesity and in fat tissue of wt mice in which obesity was induced with a high fat diet (FIG. 2a, C57,HF).

Of the Fox genes, only Foxa-2 expression was detected in adipose tissue of ob/ob mice.

Visceral and subcutaneous fat RNAs from bitt and ob/ob mice were analyzed for Foxa-2 expression by Northern blotting. The membrane was rehybridized with a probe for cyclophilin as a loading control. As shown in FIG. 1b, Foxa-2 was expressed in visceral and subcutaneous fat of ob/ob mice, but was enriched in visceral fat depots. De novo expression of Foxa-2 was specific for adipocytes and was not observed in other insulin-sensitive tissues such as muscle.

Preadipocyte (Pre) and adipocyte (Ad) protein extracts from ob/ob mice were separated by SDS-PAGE and analyzed by Western blotting for Foxa-2 and aP2 expression. As shown in FIG. 1c, both the adipocycte fraction and the stromal fraction of adipocytes containing preadipocyctes expressed the Foxa-2 protein.

Confocal image inimunostaining of visceral fat from an ob/ob mouse using anti-Foxa-2 antibodies (FIG. 1d) and TO-PRO-3 molecular probes for nuclear staining (FIG. 1e) was performed. Superimposed images are shown in FIG. 1f. As shown in FIGS. 1d-1f, Foxa-2 protein was detected in the nuclei and cytoplasm of adipocytes of obese animals.

### EXAMPLE 3

### Foxa-2 Expression in Adipocytes Correlates with Insulin Levels

Foxa-2 and Foxo-2 mRNA expression in fat of various mouse models was quantified by counting the radioactive product obtained by RT-PCR and normalizing it to the Hprt RT-PCR product. All values were calculated relative to the highest mRNA expression and correlated to plasma insulin concentration.

As shown in FIG. 2a, a striking correlation of Foxa-2 expression in fat tissue of obese mice and fasting plasma insulin levels was observed (R=0.99, p<0.0001). The correlation between plasma insulin levels and adipocyte Foxa-2 expression was markedly stronger than with Foxo-2. Foxo-2 is a forkhead transcription factor that is induced by insulin in fat cells. Cederberg et al. (2001) Cell 106:563-573.

No statistically significant linear correlation was detected betveen adipocyte Foxa-2 mRNA levels and TNF-α, leptin, triglycerides (TG), free fatty acids (FFA) or fasting blood glucose concentrations. Foxa-2 expression of ob/ob mice that were starved for 5 days decreased 2.6±0.3-fold (p<0.001) and was accompanied by lowered plasma insulin levels (19±0.7 vs. 0.7±0.1 ng/ml). The adipocyte mRNA levels of Foxa-2 of mice that lack the insulin receptor in the liver (LIRKO) and exhibit hepatic insulin resistance was also measured. These mutant mice had fasting plasma hyperinsulinemia compared to control animals (1.76±0.3 vs. 0.37+0.06 ng/ml, respectively) in the absence of obesity (body weight: 22.8 g±0.9 g vs. 23.6 g±1.1 g, epidymidal fat pad: 12±0.02 vs 0.12±0.02, Ir^{lox/lox} vs. LIRKO, respectively, LIRKO vs. Ir^{lox/lox} n=5) (Michael et al. (2000) Mol. Cell 6:87-97.) Foxa-2 expression was induced in adipocytes of LIRKO mice but was absent in Ir^{lox/lox} control mice (FIG. 2a). Furthermore, mice with adipocyte-specific insulin resistance due to the ablation of the insulin receptor in fat (FIRKO) were not hyperinsulinemic (0.19±0.02 vs. 0.27±0.07, FIRKO vs. Ir^{lox/lox}, respectively, n=5) and did not express Foxa-2 in adipose tissue. (Bluher et al. (2002) Developmental Cell 3:25-38.) These findings indicate that Foxa-2 expression in adipocytes correlates primarily with insulin levels and is not induced by tissue insulin resistance or obesity per se.

Given the strong positive correlation between serum insulin levels and adipocyte Foxa-2 gene expression, the ability of insulin per se to induce Foxa-2 expression was examined.

Primary adipocytes were isolated from lean wildtype C57B6 mice and cultured in MEM medium (control) or in the presence of insulin (100 nM; 24 hours and 60 hours, respectively) or rosiglitazone (50 μM), WY14,643 (100 μM), dexametason (1 μM), leptin (100 ng/ml), TNF-α (5 ng/ml), adiponectin (500 ng/ml) and glucagons (100 nM) for 60 hours. Gene expression was measured by semiquantitative RT-PCR. Steady state mRNA levels of Hprt were used as a control and indicate that each lane contains similar amounts of mRNA. Reactions were also assayed in the absence of reverse transcriptase, showing that mRNA was not contaminated with genomic DNA.

As shown in FIG. 2b, a strong induction of Foxa-2 was observed after 60 hours of insulin stimulation.

De novo expression of Foxa-2 could not be induced by other factors that are known to have potent effects on adipocyte differentiation and metabolism, including Ppar-α and -γ agonists, glucocorticoids, leptin, TNF-α, adiponectin, IL6, glucagon and high glucose concentrations (20 mmol/L) (FIG. 2b).

The effect of insulin on adipocyte gene expression of obese (ob/ob) animals was also analyzed.

Primary adipocytes were isolated from ob/ob mice and cultured in MEM medium (control) or in the presence of insulin (100 μM; 24 hours and 60 hours, respectively) or rosiglitazone (50 μM), WY14,643 (100 μM), dexamethason (1 μM), leptin (100 ng/ml), TNF-α, (5 ng/ml), adiponectin (500 ng/ml) and glucagons (100 nM) for 60 hours. Gene expression was measured by semiquantitative RT-PCR. Steady state mRNA levels of Hprt were used as a control and indicate that each lane contains similar amounts of rnRNA. Reactions were also assayed in the absence of reverse transcriptase, showing that mRNA was not contaminated with genomic DNA. As shown in FIG. 2c, prolonged culturing of primary adipocytes from these animals led to a strong increase in Foxa-2 expression. Ppar agonists, gluococorticoids, leptin, TNF-α, adiponectin, IL6, glucagons and glucose were unable to raise Foxa-2 expression in ob/ob adipocytes.

### EXAMPLE 4

### Foxa-2 Inhibits Adipocyte Differentiation

The physiological role of Foxa-2 was investigated by generating preadipocyte (3T3-L1) cell lines that express Foxa-1, Foxa-2 or Pref-1.

Cells were transfected with vector pcDNA3 (control) or expression vectors containing cDNAs of Foxa-1, Foxa-2 and Pref-1 under the control of a constitutive promoter. After selection with neomycin, pools of stable transfectants were induced with differentiation medium (not containing insulin). At day 8 post-induction, cells were either stained for lipid accumulation using Oil Red 0 or mRNA and total protein extracts were prepared.

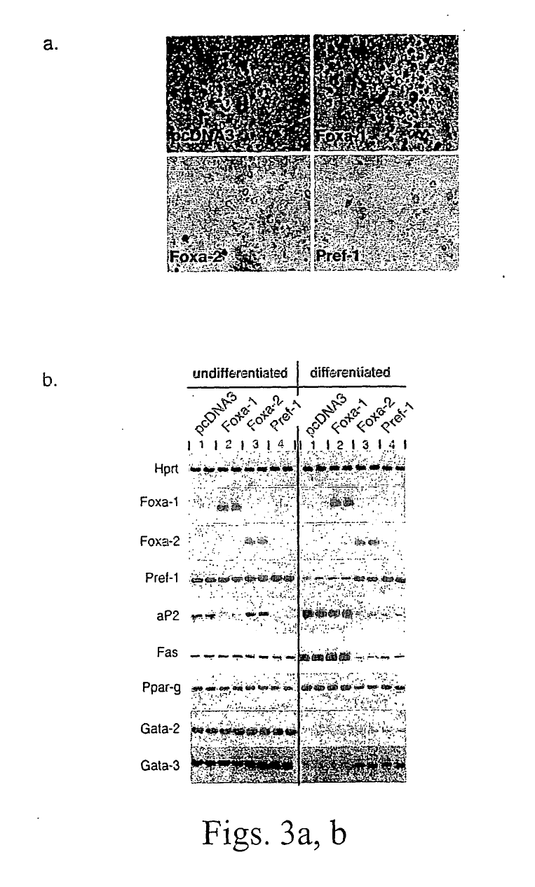

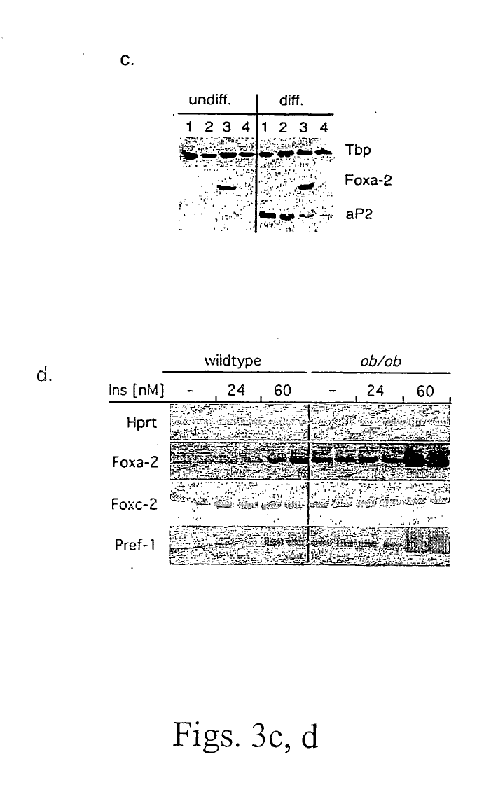

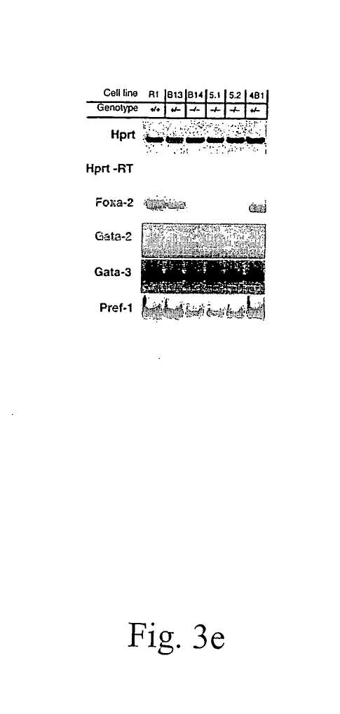

Expression of Foxa-2 or Pref-1 inhibited adipocytes differentiation in the presence of a pro-differentiation medium (FIG. 3a). In contrast, cells expressing Foxa-1 or the empty expression vector (pcDNA3) were able to accumulate lipid droplets (FIG. 3a).

Gene expression profiles were measured by RT-PCR. Hprt expression was used as a loading control indicating that each sample contained similar amounts of mRNA. No products were amplified in the absence of reverse transcriptase. Results are shown in FIG. 3b.

Western blotting was performed on cell extracts from undifferentiated and differentiated 3T3-L1 cell lines. Total protein was separated by SDS-PAGE and analyzed by immunoblotting for Foxa-2 and aP2 expression. TATA binding protein (Tbp) expression was measured as a loading control. Results are shown in FIG. 3c.

Gene expression was analyzed in a vasculo-stromal fraction (containing preadipocytes) of wildtype and ob/ob mice that were cultured in the absence (−) or presence of 100 nM insulin for 24 and 60 hours. Results are shown in FIG. 3d.

RT-PCR analysis and immunoblot analysis revealed that Foxa-1 and Foxa-2 were expressed in transfected 3T3-L1 cells but absent in untransfected cells. (FIGS. 3b and c).

Consistent with the morphological differentiation, ectopic expression of Foxa-2 prevented the down-regulation of Pref-1, Gata-2 and Gata-3, all of which have been shown to inhibit adipocyte differentiation (FIG. 3b). Smas et al. (1993) Cell 73:725-734; Tong et al. (2000) Science 290:134-138. Foxa-2 expression inhibited the induction of late markers of adipocyte differentiation such as Ppar-y, adipocyte fatty acid binding protein (aP2) and fatty acid synthase (Fas) (FIG. 3b, c).

To determine whether Foxa-2 is a direct activator of the Pref-1, Gata-2 or Gata-3 genes, the expression of these genes was compared in wt (Foxa-2^{+/+}), heterozygous (Foxa-2^{+/-}) and null (Foxa-2^{-/-}) embryonic stem cells (Duncan et al. (1998) Science 281:692-695). Wt (R1, +/+), heterozygous (B13, 4B1, +/−), and homozygous (B14, 5.1, 5.2, −/−) Foxa-2 ES cells were differentiated into EBs as described by Tong et al. (2000) Science 290:134-138 and assayed for Hprt, Foxa-2. Gata-2, Gata-3, Gata-4 and Pref-1 mRNA expression by RT-PCR. Results are shown in FIG. 3e. No differences in Gata-2 and Gata-3 gene expression were found in EBs of different Foxa-2 genotypes. In contrast, Pref-1 expression was markedly reduced in Foxa-2^{-/-} EBs compared to wt cells.

The promoter of the Pref-1 gene was analyzed to explore whether the Pref-1 gene is a direct target of Foxa-2.

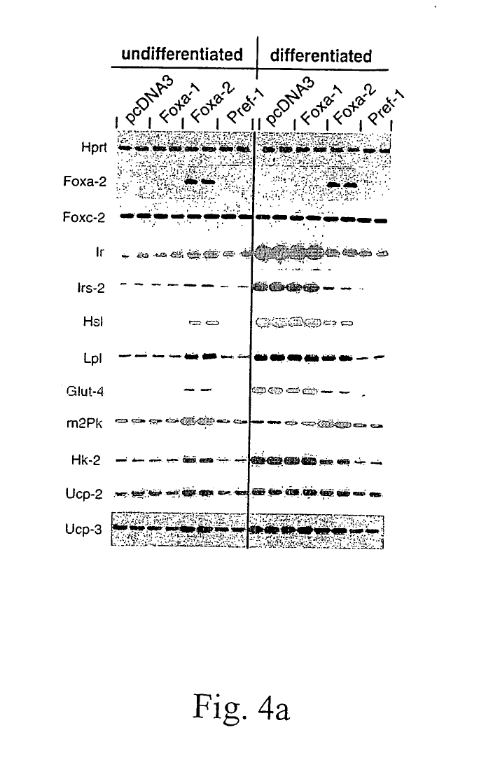

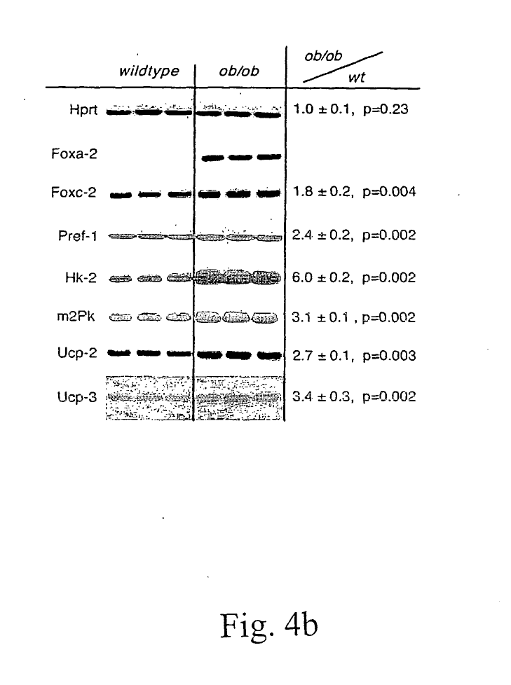

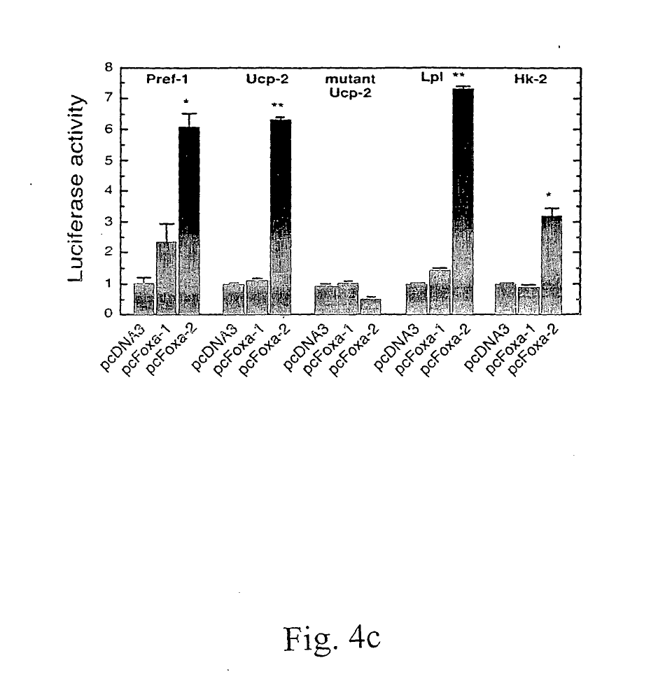

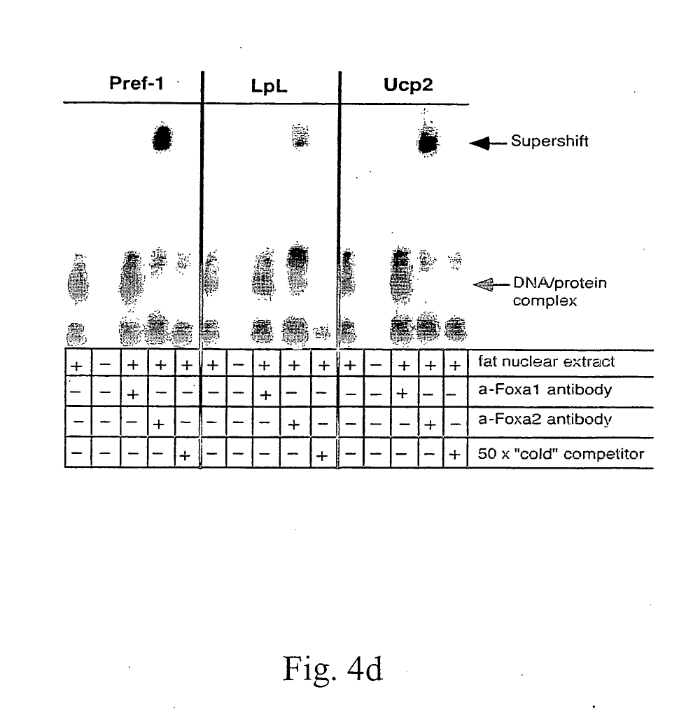

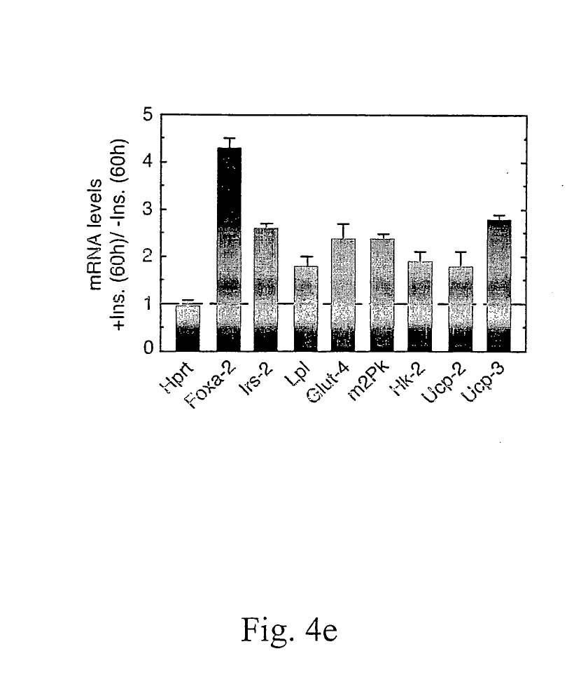

The Pref-1 transcription start site was mapped by 5′RACE and a 1.3 kb fragment of 5′-regulatory sequence was cloned into a reporter vector containing the luciferase gene (pPref-Luc). Sequence analysis of the promoter sequences identified two Foxa binding sites at position-621 and -316 that were highly conserved between mouse and human. Expression of Foxa-2 in 3T3-L1 cells in the presence of reporter construct pPref-Luc that contained a 1.3 kb promoter sequence upstream of the luciferase gene revealed a six-fold activation compared to cells that do not express Foxa-2 (FIG. 4c). Deletion of the upstream Foxa element reduced the transcriptional activity by 50%, suggesting that this element is important for Pref-1 gene expression. Constitutive Pref-1 expression markedly inhibits 3T3-L1 adipocyte differentiation (Smas et al. (1993) Cell 73:725-734) and downregulation of Pref-1 expression promotes adipogenesis (Sui et al. (2000) J. Obesity S15-S19). Furthermore, Pref-1 mutant mice have increased fat accumulation compared to wt littermates (Moon et al. (2002) Mol. Cell Biol. 22:5585-5592). Together, these data indicate that Foxa-2 inhibits adipocyte differentiation in by transcriptional activation of the Pref-1 gene.

### EXAMPLE 5

### Foxa-2 is an Insulin-Regulated Gene in Primary Preadipocytes

Stromal vascular cells from ob/ob mice are known to have increased cell replication in vitro and accumulate little triglycerides when cultured in differentiation medium containing insulin compared to lean control animals (Black et al (1995) J. Cell. Biochem 58:455-463). These data indicate that cells from obese mice are resistant to differentiation under conditions that support extensive differentiation in lean mouse cells. To test if the resistance to differentiation of obese preadipocytes may be mediated by Foxa-2, stromal vascular cells of lean and obese mice were isolated. cultured in the presence or absence of insulin, and gene expression of Foxa-2 and Pref-1 was measured. The mPNA levels were markedly higher in stromal vascular cells of ob/ob mice compared to lean littennates. The expression of Foxa-2 in stromal vascular cells of lean and obese mice could be markedly increased by insulin. However, Foxa-2 induction in ob/ob preadipocytes was profoundly higher compared to wildtype cells. Furthermore, Foxa-2 expression correlated with induction of Pref-1 gene expression. Together, these data indicate that Foxa-2 is an insulin-regulated gene in primary preadipocytes that may counter-regulate adipocyte differentiation under conditions that support extensive differentiation.

### EXAMPLE 6

### Foxa-2 is a Transcriptional Regulator in Adipocytes

Foxa proteins regulate the expression of many metabolic genes through interaction with specific binding sites in promoters/enhancers that lead to chromatin remodeling and transcriptional activation. (Chaya et al. (2001) J. Biol. Chem. 276:44385-44389; Cirillo et al. (1988) EMBO J. 17:244-254.)

To determine whether Foxa-2 is an important transcriptional regulator in adipocytes, expression analysis of genes that have putative Foxa binding sites in their 5-regulatory sequences was performed in Foxa-2 expressing preadipocytes and in differentiated adipocytes (FIG. 4a). It was found that mRNA levels of the insulin receptor (Ir), insulin receptor substrate-2 (Irs-2), hormone sensitive lipase (Hsl), lipoprotein lipase (Lpl), glucose transporters (Glut-4), muscle isoform of pyruvate kinase (m2Pk), hexokinase-2 (Hk-2) and uncoupling proteins-2/3 (Ucp-2, Ucp-3) were increased in 3T3-L1 cells that expressed Foxa-2 (FIG. 4a). The expression of Ucp-1, mitochondrial ATP-citrate lyase, glycerol 3-phosphate dehydrogenase, acyl-CoA carboxylase, Srebp-1 c, mitochondrial pyruvate decarboxylase, mitochondrial carnitine transporter, and Irs-1 were unaffected by Foxa-2. The observed expression changes were Foxa-2-specific since they are not present in preadipocytes expressing Foxa-1 (FIG. 4a). Differentiation of cells transfected with pcDNA3 (control) or expressing Foxa-1 led to a differentiation-dependent increase in the expression of Ir, Irs-2, Hsl, Lpl, Glut-4, Hk-2, Ucp-2 and Ucp-3. Foxa-2 expression blocked adipocyte differentiation and thereby, prevented the induction of these genes. These observations underline the important effect of the status of adipocyte differentiation state on gene expression. It was found that steady state mRNA levels of mPk were not affected by the differentiation state of 3T3-L1 cells and expression levels remained markedly increased in Foxa-2 expressing cells. This indicates that Foxa-2 is also an important activator of metabolic genes whose expression is not affected by adipocyte differentiation per Se.

Expression levels of these genes in adipocytes of ob/ob mice and lean littermate controls were measured by RT-PCR. Results are shown in FIG. 4b.

Each lane indicates a different animal. Semi-quantitative measurements of gene expression were obtained by densitometry, and ob/ob/wt indicates the ratio of adipocyte mRNA expression levels of the means of wt and ob/ob mice. The levels of significance of the comparison wt vs. ob/ob are shown on the right.

A striking correlation was observed between Foxa-2 expressing adipocytes of ob/ob animals and increased expression of putative Foxa-2 target genes, including Pref-1, Hk-2, m2Pk, Glut-4 and Ucp-2/3 (FIG. 4b). Moreover, primary adipocytes that were cultured in the presence of 100 nM insulin for 60 hours to induce Foxa-2 expression also showed an upregulation of these genes compared to untreated adipocytes (FIG. 4e). Gene expression was measured by semiquantitative PCR. Experiments were carried out in triplicate.

To determine whether Foxa-2 can directly activate the above-mentioned genes, the promoters were characterized. The promoters of Ucp-2, Lpl, Hk-2 and Pref-1 were cloned upstream of a luciferase reporter gene. Transcriptional activation was assayed in the absence (pcDNA3) and presence of Foxa-1 and Foxa-2 by transfecting 3T3-L1 cells with the expression vectors indicated in FIG. 4c, pCMB-β-Gal, and the luciferase reporter constructs. Luciferase activity was normalized to β-Gal activity. Each value in FIG. 4c represents the mean of 9 independent experiments±SD. As shown in FIG. 4c, Foxa-2 transactivates the promoters of Ucp-2, Lpl, Pref-1 and Hk-2 in 3T3-L1 cells.

A dose-dependent activation of all promoters was detected when co-transfected with a plasmid expressing Foxa-2. Transactivation of the reporter gene was completely lost when the Foxa binding site in the Ucp-2 promoter was selectively mutated, indicating that the Foxa binding site in the Ucp-2 promoter is functionally important (FIG. 4c).

Electrophoretic mobility shift assays were performed to determine whether Foxa-2 can bind to the putative binding sites in Ucp-2, Lpl, Hk-2 and Pref-1 promoters.

³²P-labeled probes corresponding to putative Foxa-2 binding sites in the promoters of Pref-1, Lpl and Ucp-2 vwere incubated with nuclear extracts from ob/ob fat in the presence of either unlabeled probe, anti-Foxa-1 or anti-Foxa-2 antibody. (Weinstein et al. (1994) Cell 78:578-588.) Protein/DNA complexes were separated on a 4% acrylamide gel and visualized by autoradiography. Results are shown in FIG. 4d. The radioactive probes (bottom of the gel) are not shown. *: P<0.01, **: P<0.001. Hprt: hypoxanthine phosphoribosyltransferase, Ir: insulin receptor, Irs-2: insulin receptor substrate, Hsl: hormone-sensitive lipase, Lpl: lipoprotein lipase, mPk: muscle isoform of pyruvate kinase, Hk-2: hexokinase-2, Ucp-2/3: uncoupling protein-2/3.

A major DNA/protein complex was detected with nuclear extracts from ob/ob but not wt mice (FIG. 4d). This binding activity could be competed by an unlabeled excess of ‘cold’ Foxa binding oligonucleotides. Furthermore, supershifts of the complexes were detected after preincubation of the complexes with a monospecific Foxa-2 antibody but not with an anti-Foxa-1 antiserum. The supershifts of the DNA/protein complexes were almost complete, suggesting that Foxa-2 is a major forkhead transcription factor that binds to these sites in adipose tissue of ob/ob animals.

### EXAMPLE 7

### Development of Diet-Induced Obesity and Metabolic Analysis of Pprimary Adipocyetes

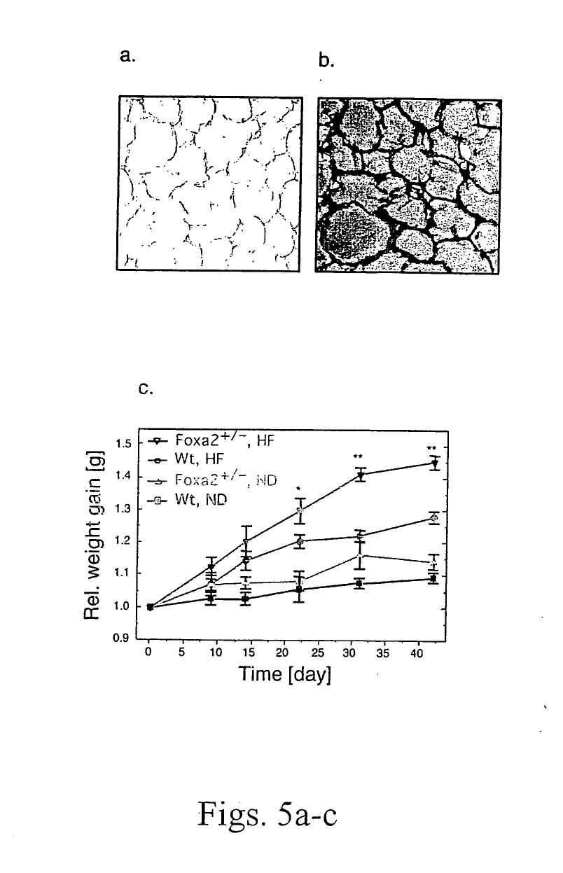

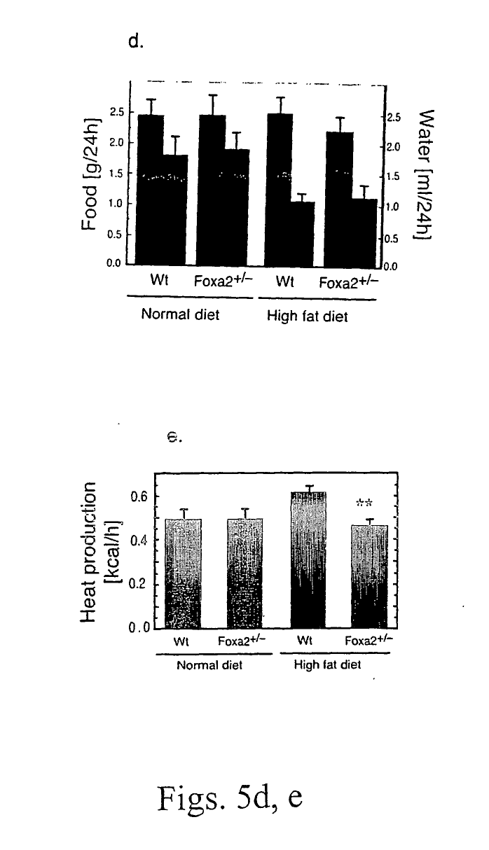

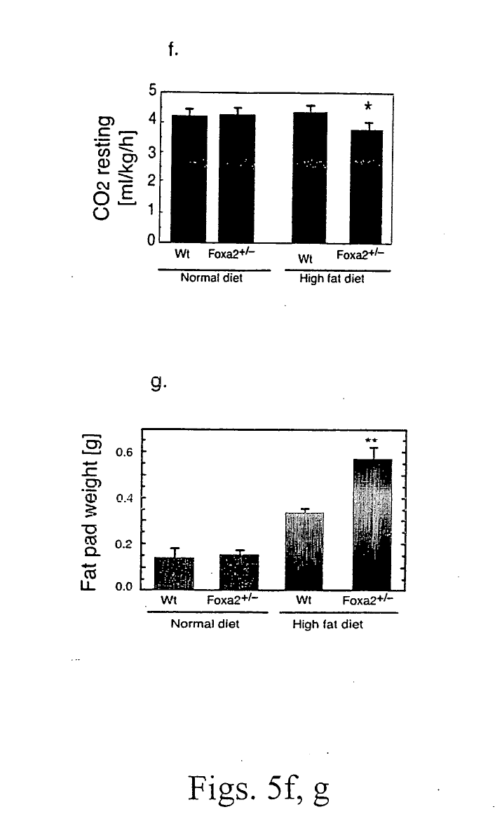

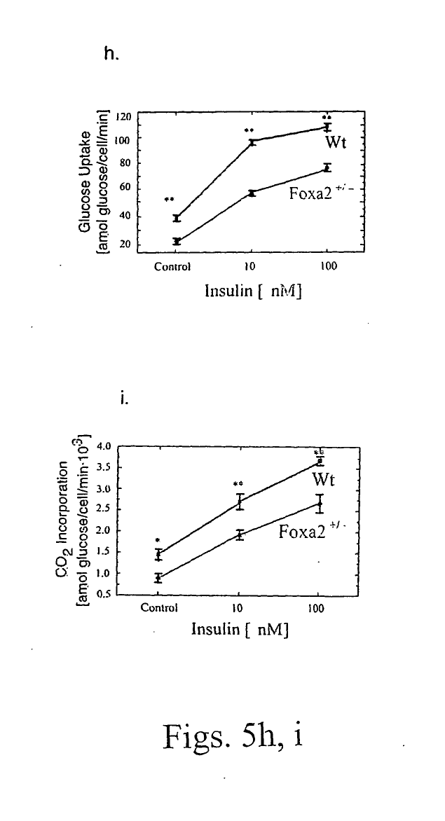

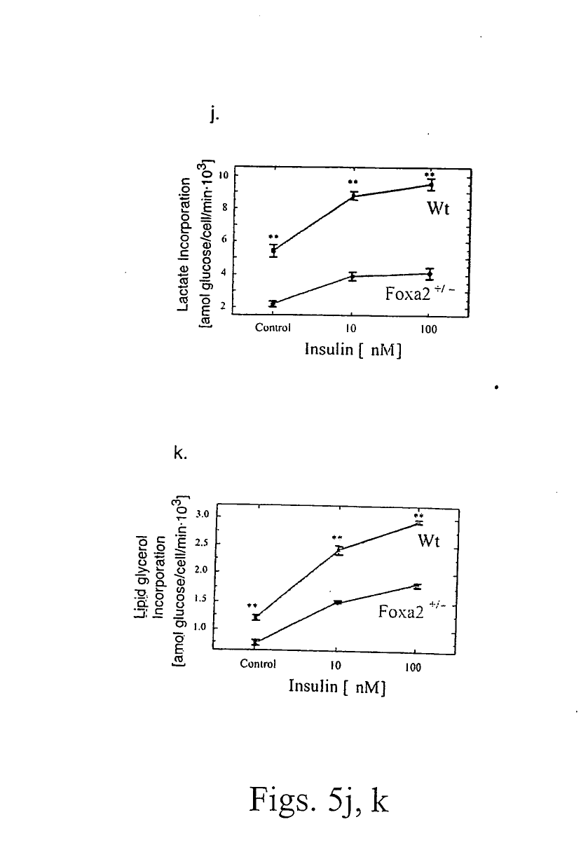

The gene expression data in the foregoing examples indicate that Foxa-2 is a powerful transcriptional activator of genes responsible for glucose uptake (Glut-4) and metabolism (Hk-2, m2Pk), insulin signaling (Ir, Irs-2), lipid metabolism (Hsl) and possibly energy dissipation (Ucp-2, Ucp-3) that can be predicted to influence adipogenesis. (Spiegelman et al. (1993) J. Biol. Chem. 268:6823-6826; Boss et al. (2000) Diabetes 49:143-156; Olefsky (1976) Endocrinology 100:1169-1177; Tozzo et al. (1997) Endocrinology 138:1604-1611.) To test this hypothesis in vivo, mutant Foxa-2 mice that have one inactivated Foxa-2 allele (Foxa-2^{+/-}) by targeted insertion of the LacZ gene were studied. (Weinstein et al. (1994) Cell 78:578-588.) Haploinsufficient Foxa-2 mice were studied because Foxa-2 null mice have an early embryonic lethal phenotype (at E7.5) and heterozygous mice exhibit normal glucose and lipid metabolism. (Ang et al. (1994) Cell 78:561-574; Shih et al. (2002) Proc. Natl. Acad Sci, USA 99:3818-3823). Foxa-2^{+/-} mice and wildtype littermates were fed a high fat (55% fat) diet and studied metabolically. RT-PCR and Xgal staining of fat from Foxa-2^{+/-} animals confirmed that these mice lacked Foxa-2 in adipocytes at the beginning of the study but induced expression during seven weeks of high fat diet (FIG. 2a, 5a, b). Fasting blood glucose, insulin, TNF-α, free fatty acid and triglyceride levels were similar between Foxa-2^{+/-} and control animals (Table 1). Mice on a high fat diet increased their fasting plasma insulin levels approximately four-fold compared to animals on a chow diet. Foxa-2^{+/-} mice exhibited a markedly increased weight gain compared to control mice when kept on a high fat diet, in spite of similar food intake and physical activity (10 g. vs. 6 g. after 42 days of high fat diet, respectively) (FIG. 5c, d). Resting heat and CO₂ production was diminished in Foxa-2^{+/-} mice, indicating that they are hypometabolic (FIG. 5e, f). Foxa-2^{+/-} animals developed noticeably increased pericardial, intrapentoneal and subcutaneous fat deposits compared to littermate animals. The adipose mass of the epidymidal fat pad was approximately double in Foxa-2^{+/-} mice compared to littermate controls after a seven-week high fat diet (FIG. 5g). The increase in adipocyte mass in Foxa-2^{+/-} mice was due to an increase in fat cell number, the size distribution of adipocytes was similar betveen mutant and wildtype animals. To test if adipocytes of Foxa-2^{+/-} mice have altered glucose metabolism, [U-¹⁴C] glucose uptake and metabolism into lactate, CO₂, lipid glycerol and fatty acids was studied. Glucose uptake and glucose incorporation into CO₂, lactate, and glyceride glycerol was strikingly reduced in adipocytes in Foxa-2^{+/-} mice compared to wildtype littermates (FIG. 5h-k). In contrast, no differences in adipocyte metabolism were observed in mutant and wildtype adipocytes of lean mice on a normal chow diet. Adipocytes of Foxa-2^{+/-} mice did not exhibit a significant reduction of glucose incorporation into fatty acids, a finding that is consistent with similar expression of genes of the fatty acid synthesis in Foxa-2^{+/-} and control adipocytes (FIG. 5l). However, reduced glycerol release from adipocytes of Foxa-2^{+/-} mice after maximal stimulation with isoproterenol and following inhibition with insulin was observed, suggesting that lipolysis is decreased in adipocytes of Foxa-2^{+/-} compared to control littermates (FIG. 5m). The defect in metabolism in Foxa-2^{+/-} adipocytes conrelated with reduced expression of Foxa-2 target genes. Steady state mRNA levels of Foxa-2, Glut-4, Hk-2, m2Pk, Irs-2, Ucp-2 and Ucp-3 were reduced ˜50% and more in fat cells of diet-induced obese Foxa-2^{+/-} compared to wildtype mice (FIG. 5n). Together, these data demonstrate that Foxa-2 is an important metabolic regulator of glucose metabolism and energy dissipation in adipocytes of hyperinsulinemic obese mice.

FIGS. 5a and b show X-gal staining of adipose tissue of wildtype (a) and Foxa-2^{+/-} (b) mice after a 7-week high fat diet. FIGS. 5c-g show relative weight gain (c), food and water intake (d), heat production (e), resting CO₂ production (f), and epidymidal fat pad weight (g) of Foxa-2^{+/-} and wildtype littermates on chow and high fat diets. FIGS. 5h-l show glucose metabolism into different pathways at 10 and 100 nM insulin in isolated adipocytes from Foxa-2^{+/-} (red) and wildtype (black) littermates. [U-¹⁴C]Glucose uptake in isolated adipocytes from epidymidal fat (h), [U-¹⁴C]Glucose incorporation into CO₂ (l), lactate (j), lipid glycerol (k), and fatty acids (l), measured after 2 hours incubation in the absence (control) or presence of insulin. FIG. 5m shows glycerol release from adipocytes in the presence or absence of insulin after stimulation of lipolysis with isoperenterol (Isop). FIG. 5n shows measurements of relative gene expression levels of metabolic genes in adipocytes of Foxa-2^{+/-} and wildtype littermates (100%) using semiquantitative RT-PCR. All mice were female, weeks of age, n=4, means±SD. *: P≦0.0S, **: P≦0.01, ***: P≦0.001, ****: P≦1×10⁻⁵, ns: not significant.

Table 1 below shows plasma levels of insulin, glucose, cholesterol, free fatty acid (FFA), triglycerides (TG), leptin and TNF-α of lean wildtype (C57) and mutant Foxa-2 (Foxa-2^{+/-}) mice on a normal (ND) or high fat diet (HF). Data are firom n=5 in each group, age: 8 weeks old female mice, duration of diet: 42 days.

TABLE 1

| | Insulin | Glucose | TNFα | Cholesterol | Leptin | FAA | TG |
|---|---|---|---|---|---|---|---|
| | ng/ml | mg/dl | pg/ml | mgl/dl | Eq/L | mmol/L | mg/dl |
| C57 | 0.42 ± 0.02 | 96 ± 35 | < 10 | 43 ± 5 | 0.6 ± 0.1 | 0.15 ± 0.02 | 52 ± 5 |
| C57, HF | 1.63 ± 0.08 | 96 ± 24 | 12.9 ± 0.8 | 66 ± 5 | 0.6 ± 0.1 | 0.16 ± 0.04 | 62 ± 11 |
| Foxa-2^{+/-} | 0.50 ± 0.06 | 84 ± 18 | < 10 | 38 ± 11 | 0.8 ± 0.1 | 0.18 ± 0.04 | 54 ± 11 |
| Foxa-2^{+/-},HF | 1.52 ± 0.03 | 87 ± 8 | 12.3 ± 1.1 | 54.9 | 0.8 ± 0.1 | 0.21 ± 0.08 | 80 ± 14 |

### EXAMPLE 8

### Expression of Fxr-1 is Positively Regulated by Foxa-2

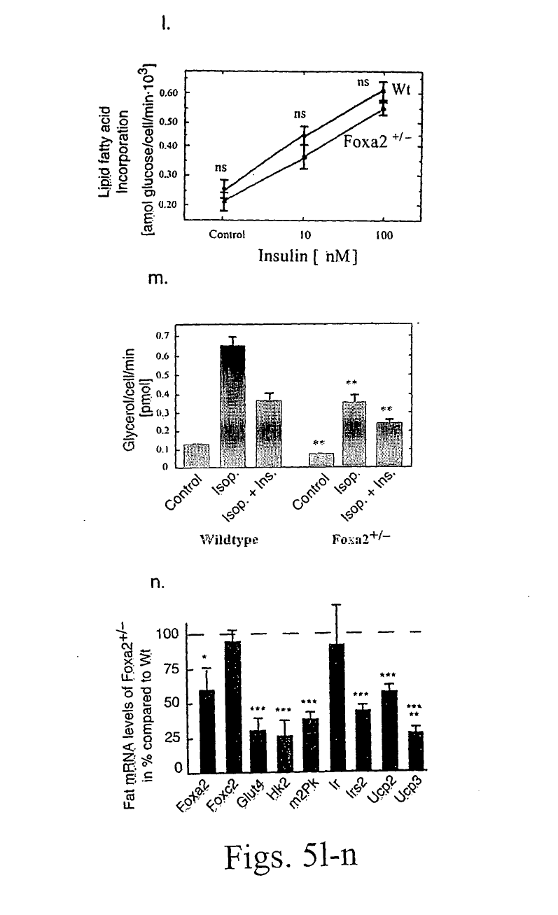

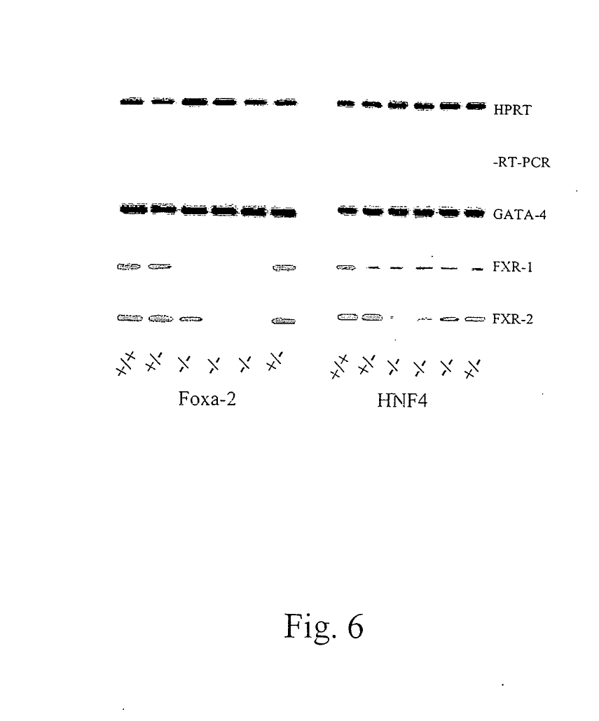

Expression of murine Fxr-1 and Fxr-2 in differentiated ES cells (embryoid bodies, Ebs) deficient for Foxa-2 was assessed. Wildtype (+/+), heterozygous (+/−) and null (−/−) Foxa-2 ES cells were differentiated and expression levels were analyzed for Gata-4 (a marker for visceral endoderm), Fxr-1 and Fxr-2. As shown in FIG. 6, Fxr-1 expression is absent in cells lacking Foxa-2 expression.

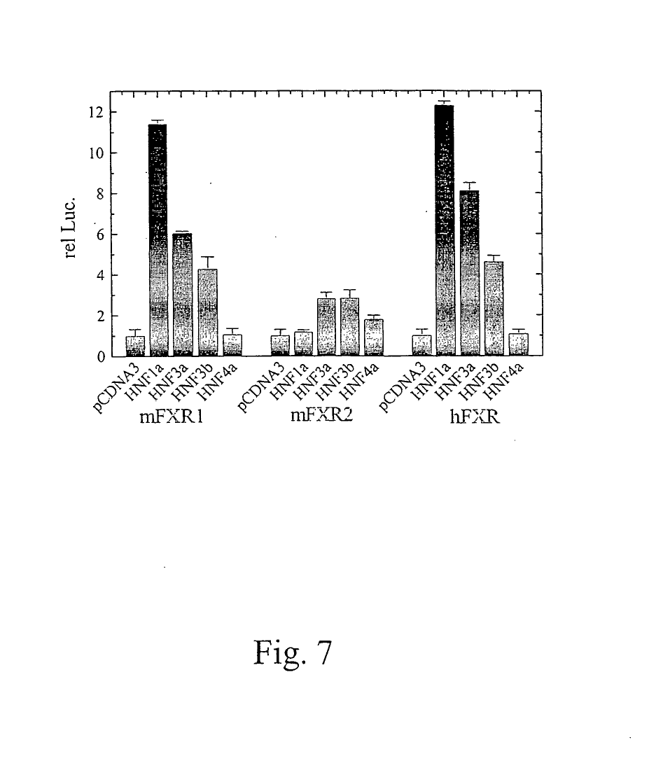

Murine Fxr-1 and human Fxr promoters were analyzed as follows. HepG2 cells were transfected with vectors expressing the transcription factors indicated in FIG. 7 and with a reporter construct in which the mouse (mFxr-1 or mFxr-2) or human (FXR) promoters are upstream of the luciferase gene. Constructs were cotransfected with CMV-Xgal vector to normalize transfection efficiencies. Luciferase activity was measured 48 hours after transfection. A conserved Foxa binding site was identified in the mouse Fxr-1 and human FXM promoters. As shown in FIG. 2, this promoter can be activated when coexpressed with Foxa-2.

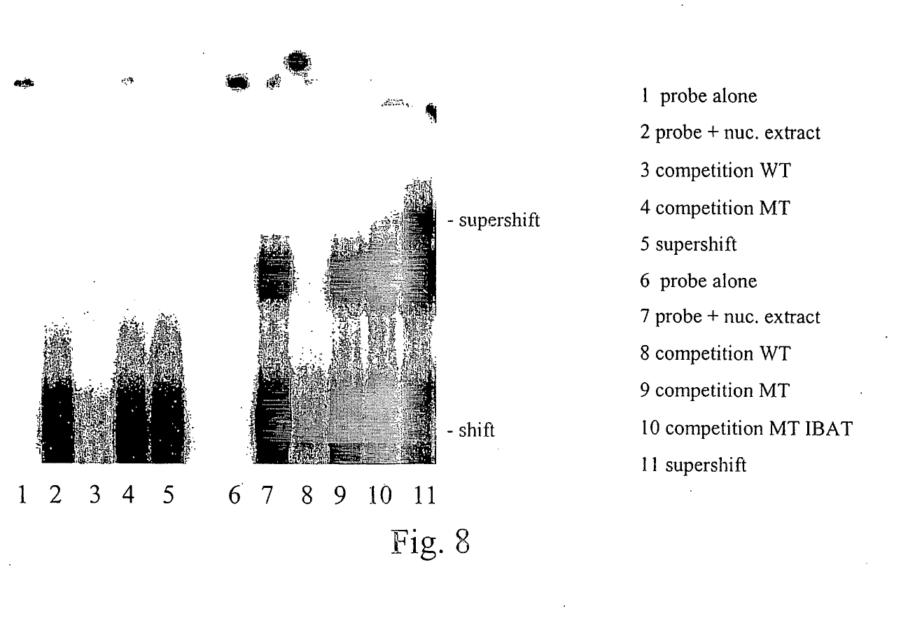

Electrophoretic mobility shift analysis (EMSA) of the Foxa-binding site in the Fxr-1 promoter was performed. As shown in FIG. 8, lanes 1-5, gel shift analysis with a putative HNF-4 binding site in the Fxr-1 promoter exhibited no binding. As shown in FIG. 8, lanes 6-11, EMSA with a putative Foxa binding site in the Fxr-1 promoter showed that Foxa-2 protein binds to a consensus sequence in the murine Fxr-1 promoter.

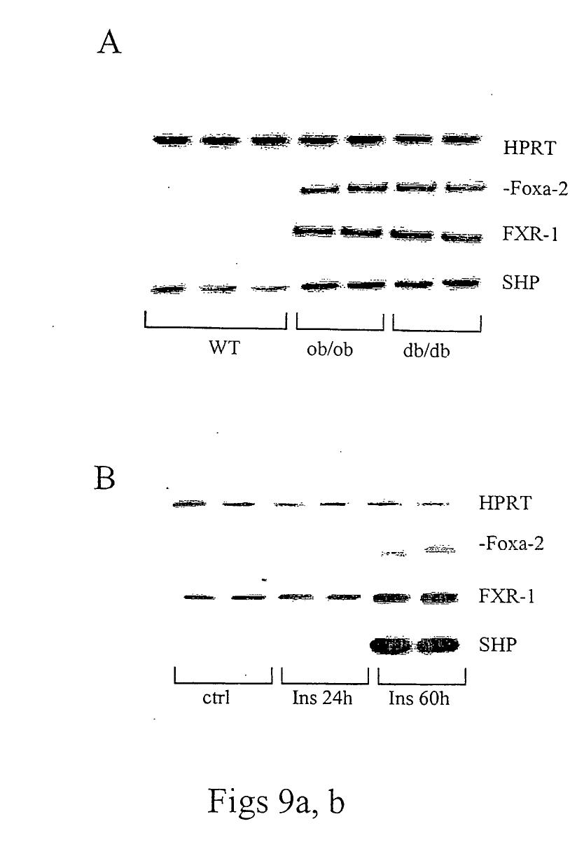

It was also demonstrated that, like Foxa-2, Fxr is expressed de novo in adipocytes of obese (hyperinsulinemic) mice. FIG. 9a shows de novo expression of Fxr-1 in adipose tissue of ob/ob and db/db mice.

It was further demonstrated that Fxr expression can be induced by culturing primary adipocytes of lean mice in the presence of insulin (50 nM) for 60 hours. Expression of Shp, a known target gene of Fxr, is also induced in insulin treated cells. Results are shown in FIG. 9b.

These data indicate that expression of Foxa-2 in adipocytes of obese animals activates Fxr and Shp expression.

### EXAMPLE 9

### Materials and Methods

The following materials and methods were used in Examples 10-12.

Materials. Insulin was from Sigma, Ly294002 and PD 98059 were from Calbiochem.

Generation of Plasmids. Expression vectors for Foxa-1 and Foxa-2 were generated by cloning the coding region of either rat Foxa-1 or rat Foxa-2 into pcDNA3 either with or without fusion to an N-terminal Flag/HA-tag. Mutants (TI56A and R153K) were generated by PCR mutagenesis using the Quickchange protocol (Invitrogen). Expression vectors for HA-Akt1 (pCMV-HA-Akt) were generated by cloning the coding region of human Akt1 into pcDNA3 fused to an N-terminal HA-tag. The vectors encoding the HA-tagged forms of constitutively active Akt (pCMV-HA-myrAkt) and inactive Akt (pCMV-HA-Akt1_{K179A}) are as described by Cross et al. (1995) Nature 378: 785-789. Bacterial expression vectors of Foxa-2 and Foxa-2_{T156A} were generated by cloning the cDNA into pGEX-4T2 (Pharmacia).

Cell Culture. HepG2 and HEK/293 cells were maintained in DMEM supplemented with 4.5 g/l glucose, 10% fetal calf serum, 2 mM glutamine; 50 μg/ml gentamycin/streptomycin in a humidified incubator at 5% CO₂.

Transfections and Transactivation assay. HepG2 cells were grown to 60-70% confluence in a 24-well dish and transfected with 50 ng of each reporter gene (p6xCdx-TkLuc or pPEPCK-Luc), pCMV-β-Gal as internal reference and the expression vectors for wildtype or mutant Foxa-1 and Foxa-2 and human Akt1/Akt2 using the transfection reagent Fugene6 (Roche). Cells were grown for 48 hours and luciferase activity was measured using the Luciferase Detection System (Promega). Luciferase was normalized for transfection efficiency by β-galactosidase activity. HEK/293 cells were grown to 80% confluence in a 100 mm cell culture dish and transfected with 10 μg of each expression vector for mutant or wlildtype Foxa-2 and human Akt1 or Akt2 using Lipofectamine 2000 (Invitrogen).

Expression and Purification of Recombinant GST-Foxa-2. BL21 E. coli cells were grown to an OD₆₀₀ of 0.8 and protein expression was induced by addition of 0.4 mM IPTG. Cells were harvested by centrifugation and lysed in 10 mM Tris/HCl, pH 7.4, and 30 mM NaCl by sonication at 4° C. Soluble E. coli proteins were equilibrated to 10 mM Tris/HCl, pH 7.4, 30 mM NaCl and chromatographed on a Mono-Q column (1×5 cm, 2 ml/min) using an FPLC system (Pharmacia). GST-Foxa-2 and GST-Foxa-2_{T156A} eluted at approximately 300 mM NaCl. Fractions containing GST-Foxa-2 were pooled and concentrated using the Centriprep system with 10 kDa cut off (Millipore). The concentrated solution was subjected to gel filtration on Supprose 12 (1.8×60 cm, 0.4 ml/min) in 10 mM Tris/HCl, 250 mM NaCl at pH 7.4. Purity of the protein was determined by SDS-PAGE.

Immunoprecipitation. Foxa-2 was precipitated from cell lysates using polyclonal anti-Foxa-2 antibodies (Ruiz i Altaba et al. (1993) Mech. Dev. 44: 91-108) bound to gamma-bind-sepharose (Pharmacia) overnight at 4° C. Akt was precipitated from cell lysates using monoclonal anti-HA antibody (Sigma) bound to gamma-bind-sepharose (Pharmacia) for 2 hours at 4° C. After washing of the precipitate the proteins were eluted with SDS-loading buffer, separated by SDS-PAGE (12%), and analyzed by Western blotting using either monoclonal anti Foxa-2 antibody (1:4000) or polyclonal anti-HA antibody (1:2000) (Sigma) and respective secondary antibodies linked to horseradish peroxidase (Calbiochem). Proteins were visualized by chemoluminescence detection using the ECL system (NEN).

In vitro kinase assay. Phosphorylation of Foxa-2 was analyzed using an iin vitro kinase assay. Akt was precipitated from 200 μg of total protein lysate from HEK/293 cells transfected with pCMV-HA-Akt or pCMV-HA-LCK-Akt using anti HA antibody (Sigma) bound to gamma-bind-sepharose (Pharmacia) for 2 hours at 4° C. Precipitates were washed 3 times with kinase buffer (25 mM MOPS pH 7.4, 25 mM β-glycerophosphate, 20 mM MgCl₂, 2 mM MnCl₂, 1 mM DTT, supplemented with protease inhibitor cocktail (Roche)) and incubated with 5 μg of either purified Foxa-2 and Foxa-2_{T156A} or with GST-Akt (positive control) in the presence of 0.5 μCi γ-ATP for 15 min at 37° C. Proteins were eluted with SDS-loading buffer, separated by SDS-PAGE (13.5%), and analyzed by autoradiography. Equal loading was confirmed by analyzing the expression levels of Foxa-2 by Western blotting.

Immunofluorescence microscopy. Cells were fixed for 30 minutes at room temperature with 2% paraformaldehyde. For immunofluorescent detection of Foxa-1 or Foxa-2, fixed cells were incubated with respective polyclonal antibodies (1:100) (Ruiz i Altaba et al. (1993) Mech. Dev. 44: 91-108) overnight at 4° C. After washing, the cells were treated with anti rabbit IgG secondary antibody linked to Alexa Fluor 488 (Molecular Probes). Immunofluorescent staining was visualized using laser-scanning micrsocopy.

Electrophoretic mobility shift assay. Whole cell extracts from transfected HEK/293 cells (20 μg) were incubated with a ³²P-labeled double-stranded oligonucleotide probe with the Foxa binding sites of the Igfbp-1 promoter (Allander et al. (1997) Endocrinology 138: 4291-4300). The reaction was performed in a mixture containing Hepes buffer (20 mM, pH 7.9), KCl (40 ml), MgCl₂ (1 mM), EGTA (0.1 mM), DTT (0.5 mM), 4% Ficoll and poly(dIdC) at RT for 15 min. Competition analysis was performed by incubating the cellular extracts and the probe with the unlabeled oligonucleotide for a consensus Foxa binding site. Supershift analysis was carried out by incubating the nuclear extracts with either anti-Foxa-1 or anti-Foxa-2 antibody. The reaction mixture was loaded on a 6% non-denaturing polyacrylamide gel in TBE buffer (0.023 M Tris-borate, 0.5 mM EDTA) and run at 4° C. Bands were visualized by autoradiography.

### EXAMPLE 10

### Insulin Dependent Decrease of Foxa-2 is Mediated by PI3-Kinase-Akt

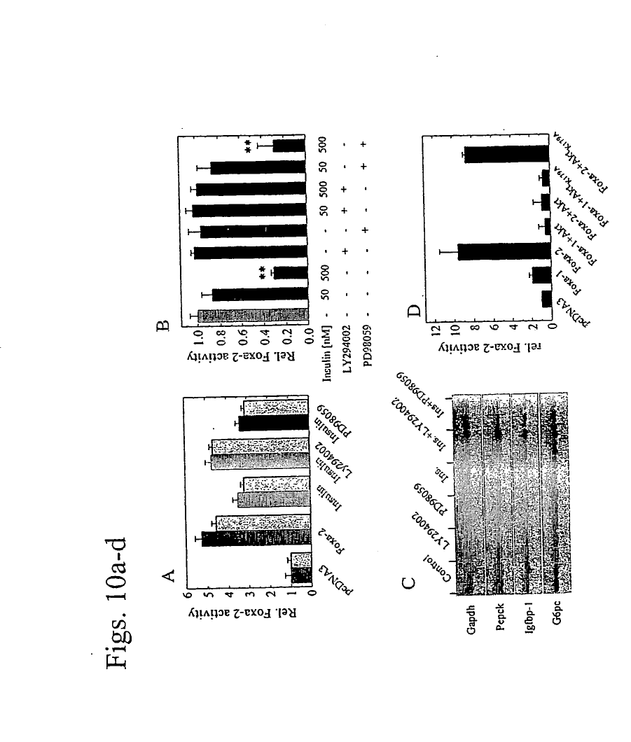

To analyze whether Foxa-2 activity is regulated by insulin, HepG2 cells were transfected with a Foxa-2 expression vector and either plasmid pPepck-Luc or p6xCdx-TkLuc that contain a 621 bp promoter framnent of the human PEPCK gene or six Foxa-2 binding sites of the Cdx-2 gene upstream of a minimal promoter and the luciferase gene, respectively. Cotransfection of Foxa-2 with both reporter constructs led to an approxirnately five-fold increase in activity compared to control transfection. Treatment of the cells with insulin (100 nM) for the duration of transfection significantly decreased Foxa-2 activity. Coincubation with the PI3-kinase inhibitor Ly294002 (10 μM) prevented insulin mediated decrease of Foxa-2 activity, while the MAPKK1 inhibitor PD98059 (10 μM) did not influence insulin regulation of Foxa-2 activity (FIG. 10a).

To determine whether insulin decreases endogenous Foxa-2 activity in HepG2 cells, cells were transfected with the reporter construct alone and Foxa-2 activity was measured after stimulation with insulin and PI3-kinase or MAPKK1 inhibitors. Insulin stimulation led to a dose-dependent decrease of Foxa-2 activity (80% decrease at 500 nM insulin). This decrease in activity was ablated when cells were coincubated with Ly294002 but not with PD98059 (FIG. 10b).

Levels of mRNA were analyzed to determine whether inhibition of Foxa-2 target gene expression by insulin is controlled at a transcriptional or posttranscriptional level. The mRNA levels of Pepck, G6pc and Igfbp-1 in primary hepatocytes that were cultured either in the presence or absence of insulin, PI3-kinase or MAPKK1 inhibitors were measured. The mRNA levels were significantly reduced in insulin or insulin/PD98059 treated hepatocytes but not in controls (no insulin) or insulin/Ly294002 treated cells (FIG. 10c). The reduced expression could not be attributed to increased expression levels of Foxa-1, 2 and 3, since expression levels of these genes did not significantly change in insulin-treated cells (FIG. 10c). These data support previous reports by O'Brien et al. (1995) Mol. Cell Biol. 15: 1747-1758, indicating that insulin can inhibit the expression of Foxa target genes in hepatocytes, and that the rapid insulin-mediated reduction in transcriptional activity is unlikely due to alterations in expression levels of Foxa-1-3.

The demonstration that insulin signaling leads to a PI3-kinase mediated decrease in Foxa-2 activity led to an investigation of whether downstream targets of PI3-kinase are involved in the modulation of Foxa-2 activity. One prominent downstream target of PI3-kinase, which has been shown to modulate target gene activity by phosphorylation, is Akt (Datta et al. (1999) Genes Dev. 13: 2905-2927). To assess the involvement of Akt in mediating the insulin dependent decrease in Foxa-2 activity, HepG2 cells were transfected with expression vectors Foxa-1 or Foxa-2 and expression vectors for Akt1/2 or an inactive form of Akt (Akt1_{K179A}). Cotransfection of Foxa-2 with Akt1 or Akt2 completely abolished Foxa-2 activity while expression of the inactive Akt_{K179A} protein had no effect (FIG. 10d).

The Foxa-2 primary structure was analyzed for potential Akt phosphorylation sites Alessi et al. (1996) FEBS Lett. 399: 333-338. A putative Akt tyrosine phosphorylation site (RRSYTH) was identified in the human Foxa-2 protein at position aa152-157 that was completely conserved between human, mouse, rat, chicken, X. laevis, C. elegans and S. pombe. No Akt phosphorylation consensus sequences were detected in either Foxa-1 or Foxa-3 (FIG. 11a).

To determine whether the identified site was responsible for the Akt mediated regulation of Foxa-2 phosphorylation, the following experiment was performed. Two different mutants of Foxa-2 were generated: Foxa-2_{T156A} which cannot be phosphorylated, and Foxa-2_{R153K}, a mutant that is unable to bind to Akt (Alessi et al. (1996) FEBS Lett. 399: 333-338). HepG2 cells were cotransfected with p6xCdx-TkLuc, expression vectors for either wt Foxa-2, Foxa-2_{T156A} or Foxa-2_{R153K}, together with pCMV-HA-Akt-1 or -2. A dose-dependent inhibition of Foxa-2 activity was observed when transfected with increasing amounts of the Akt1 or Akt2 expression vectors (FIG. 11b). This decrease was not observed in cotransfections with either Foxa-2_{T156A} or Foxa-2_{R153K}, indicating that the identified site is responsible for Akt-mediated regulation of Foxa-2 activity. The effect of wt and mutant Foxa-2 proteins on the activity of Pepck gene transcription was analyzed by coexpressing these proteins together with pPepck-Luc. Transcriptional activity of the Pepck promoter was increased about 6-fold in cells cotransfected with Foxa-2. A dose-dependant decrease was observed when increasing amounts of Akt1 or Akt2 expression vectors were cotransfected. No decrease in activity was observed in transfection with either Foxa-2_{T156A} or Foxa-2_{R153K} using increasing amounts of Akt (FIG. 11c).

### EXAMPLE 11

### Akt Interacts with and Phosphorylates Foxa-2 at PositionTI56

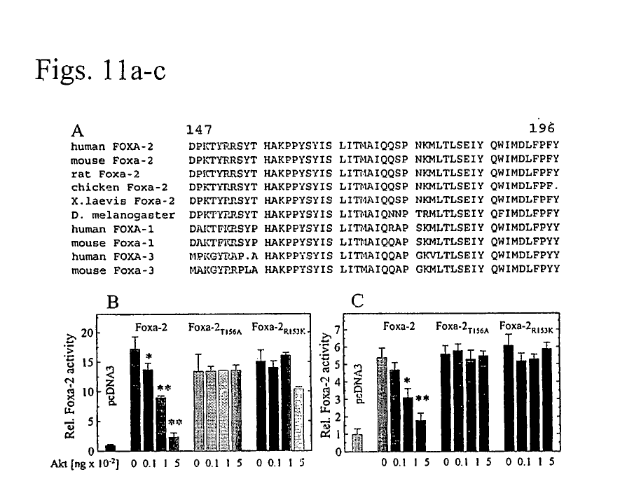

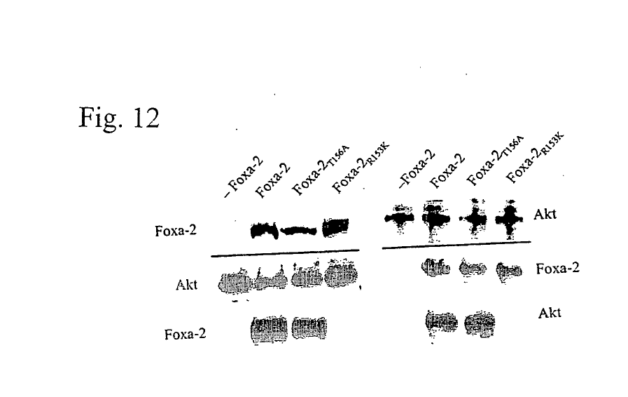

To demonstrate that Akt modulates Foxa-2 activity by direct interaction with the putative Akt phosphorylation site, immunocoprecipitation experiments were performed. HEK/293 cells were transfected with Foxa-2, Foxa-2_{T156A}, or Foxa-2_{R153K} and HA-Akt. Foxa-2 was precipitated using a polyclonal anti-Foxa-2 antibody, HA-Akt was precipitated with a monoclonal anti-HA antibody. The precipitates were separated by SDS-PAGE and analyzed by Western blotting. As can be seen in FIG. 3 precipitation of HA-Akt led to coprecipitation of Foxa-2 and Foxa-2_{T156A} but not of Foxa-2_{R153K}. Comparison of Foxa-2 and Foxa-2_{T156A} showed that interaction of Foxa-2 with A-kt is approximately 2-fold weaker than wildtype Foxa-2. Conversely, precipitation of Foxa-2 yielded similar results, as Akt could be coprecipitated together with Foxa-2 and Foxa-2_{T156A} but not with Foxa-2_{R153K}. No differences in interaction of Foxa-2 and Foxa-2_{T156A} could be seen using this approach (FIG. 12).

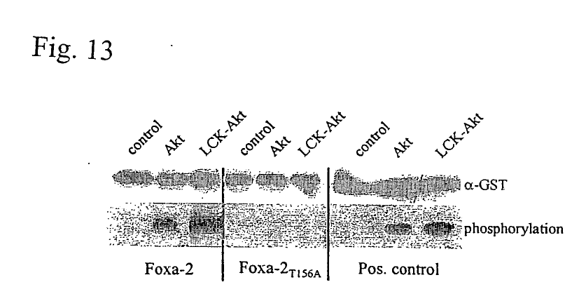

To confirm that Foxa-2 can be phosphorylated by Akt kinase activity, the following experiment was performed. Recombinant GST-Foxa-2 and GST-Foxa-2_{T156A} proteins were expressed in E. coli BL21 cells and the soluble protein was purified by anion-exchange chromatography and subsequent size exclusion chromatography. Active Akt was purified from transfected HEK/293 cells by immunoprecipitation with anti HA-antibody. GST-Foxa-2, GST-Foxa-2_{T156A} or GST-Akt (GST fused to a consensus Akt phosphorylation site) (Vandromme et al. (2001) J. Biol. Chem. 276: 8173-8179) as positive controls were coincubated with either control cell precipitates (untransfected cells), precipitated HA-Akt or precipitated constitutive active Akt in the presence of 0.5 μCi [γ^{32P}]-ATP. FIG. 4 shows that wildtype Foxa-2 was phosphorylated by either Akt or myrAkt, while no phosphorylation could be observed using Foxa-2_{T156A} protein as substrate. Equal loading of wildtype Foxa-2 and Foxa-2_{T156A} was demonstrated by Western blotting of the phosphorylation reactions (FIG. 13).

### EXAMPLE 12

### Foxa-2 Phosphorylation by Insulin-PI3-Kinase-Akt Signaling Leads to Nuclear Export

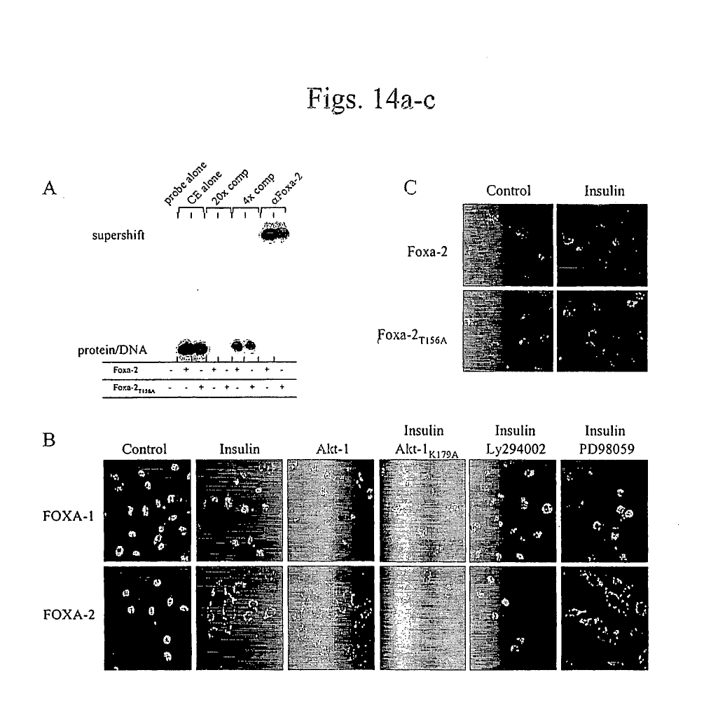

The mechanisms underlying the inhibitory effect of Foxa-2 phosphorylation on the transcriptional activation of target genes were examined. Mechanisms that may account for the inhibitory effects of Akt on Foxa-2 function include an Akt induced reduction of total Foxa-2 expression levels, impairment of binding to DNA, impairment of Foxa-2's intrinsic transcriptional activation or repressor function or by changes in Foxa-2's nuclear localization. It was found that expression of Akt did not significantly change mRNA or protein expression levels of Foxa-2 in HepG2 cells. The DNA binding activity of nuclear extracts from HepG2 cells transfected with either wildtype or Foxa-2_{T156A} expression vectors was compared. Electrophoretic mobility shift assays were performed to investigate if mutant Foxa-2 proteins can bind to a Foxa binding site of the Igfpb-1 promoter (Allander et al. (1997) Endocrinology 138: 4291-4300). Wildtype and phosphorylation deficient mutant Foxa-2_{T156A} bound equally to ³²P-labelled oligonucleotide probes that contained the Foxa binding site. These data indicate that the phosphorylation state of Foxa-2 does not lead to impairment in DNA binding (FIG. 14a).

To determine whether Akt-induced phosporylation of Foxa-2 has an effect on the subcellular distribution of this transcription factor, the following experiment was performed. HepG2 cells were grown to 60% confluency and endogenous Foxa-1 and Foxa-2 proteins were visualized by immunofluorescence after staining with anti Foxa-1 and Foxa-2 antibodies. Cells were either examined in the absence or presence of insulin (50 nM) and/or Ly294002 or PD98059, and after transfection with either Akt1 or Akt2 expression vectors (FIG. 5B). When the endogenous PI3-kinase-Akt pathway was inhibited (by serum starvation (Control) or treatment with Ly294002 in the presence of insulin) the endogenous Foxa-2 protein was localized almost exclusively in the nucleus. In contrast, cells in which the PI3-kinase-Akt pathway was activated by either treatment with insulin or by overexpression of Akt1/2, Foxa-2 was efficiently excluded from the nucleus and largely detected in the cytoplasm (FIG. 14b). Treatment of cells with MAPKK1 inhibitor PD98059 had no effect on insulin stimulated nuclear exclusion of Foxa-2. The subcellular distribution of Foxa-2 in cells that were stimulated with insulin and expressed a dominant negative form of Akt (Akt_{K179A}) were resistant to nuclear exclusion. In contrast to the drastic changes in subcellular localization of Foxa-2 upon stimulation of the PI3-kinase-Akt pathway, Foxa-1 protein was not responsive to the activation of this pathway. The same results were obtained when using a glucose responsive pancreatic β-cell line (Min6). To examine if the effect of Akt on the subcellular localization of Foxa-2 was due to the phosporylation of TI56 residue, wvt and mutant Foxa-2_{T156A} HA-tagged protein were expressed in HepG2 cells. The intracellular distribution of this protein in the presence and absence of insulin was examined. In contrast to the wildtype Foxa-2 protein, Foxa-2_{T156A} was exclusively localized in the nucleus after activation of the PI3-kinase-Akt pathway with insulin (FIG. 14c). Together, these data demonstrate that insulin stimulation induces phosphorylation via the endogenous PI3-kinase-Akt pathway of a conserved residue, specific for Foxa-2, and that this site plays a crucial role in sequestering Foxa-2 in the cytoplasm, thereby inhibiting Foxa-2's ability to activate transcription of target genes in the nucleus.

### EXAMPLE 13

### Methods

The following methods were used in Examples 14-18.

Animal and Metabolic Cage Studies

All animal models were maintained in C57B1/6J background and maintained on a 12 hours light/dark cycle in a pathogen-free animal facility. Groups of animals were fed a high fat diet (Harland Teklad) containing 50% fat for 6 or 12 weeks. Oxygen consumption, CO₂ and heat production and food and water intake were simultaneously determined for 4 mice per experiment in an Oxyniax metabolic chamber system (Columbus Instruments, Columbus, Ohio). Individual mice were placed in a chamber with an airflow of 0.6 L/min and one reading per mouse was taken at 4-min intervals over 24 h. Resting metabolic parameters were determined by integrating values at periods of no activity.

Adenovirus Generation

Adenoviruses were generated using the Rapid Adenovirus Production System (Viraquest), employing the pVQ CMV K-Npa shuttle vector. Viruses were designed to express GFP from an independent promoter, in addition to Foxa-2 or Foxa-2_{T156A} (Ad-Foxa-2 and Ad-Foxa-2TI56A, respectively). For in vivo experiments, mice were injected with 1×10¹¹ particles of adenovirus. Empty virus expressing only GFP served as control (Ad-GFP).

Generation of Anti-Phospho Peptide Antibodies

Polyclonal antibodies were produced by immunizing rabbit with a synthetic phosphorylated peptide (KLH coupled) corresponding to residues surrounding Thr156 of human Foxa-2. Antibodies were purified by protein A and peptide affinity chromatography.

Electrophoretic Mobility Shift Assay

Nuclear extracts from livers, prepared according to Stuempfle et al. (1996) Biotechniques 21:48-50 (20 μg), were incubated with ³²P-labeled double-stranded oligonucleotide probes homologous with either the Foxa-2 binding site from the IGFBP promoter (Unterman et al. (1994) Biochem Biophys. Res. Commun. 203:1835-41) or the HNF1a binding site from the SHP promoter. (Shih et al. (2001) Nat. Gene. 27:375-382). Competition analysis was performed by incubating the cellular extracts and the probe with the non labeled oligonucleotide. Supershift analysis was carried out by incubating the nuclear extracts with anti-Foxa-2 (Ruiz i Altaba et al. (1993) Mech. Dev. 44:91-108 or anti-HNF1α antibody (Cell Signaling).

Immunoblotting and Immunobistochemistry

Cytosolic and nuclear protein extracts were separated by SDS-PAGE (11.5%) and transferred onto a nitrocellulose membrane (Schleicher&Schuell) by electroblotting. Foxa-2 was detected with anti-Foxa-2 antiserum (1:1000), Foxo-1 was detected using affinity purified antibody (Cell Signaling) (1:1000). Membranes were incubated with primary antibodies overnight at 4° C. Incubations containing the secondary antibody were perfoimed at RT for 1 hr.

Cryosections of livers (4 μM) were fixed for 15 min at RT with 4% paraformaldehyde. For immunofluorescent detection of Foxa-2 or HA-Foxa-2, fixed sections were incubated with either anti-Foxa-2 antibody (1:100) or anti-HA antibody (Convance) (1:1000) overnight at 4° C. After washing, sections were treated with secondary antibody linked to Alexa Fluor 488 (Molecular Probes). For nuclear counterstaining, TOPRO-3 dye (Molecular Probes) was used. Immunofluorescent staining was visualized using laser-scanning micrsocopy.

Reverse Transcriptase-PCR and Affymetrix Gene Array

Total RNA was extracted from liver using Trizol following manufacturer's instructions (Life Teclmologies, Inc.). Contaminating genomic DNA was removed by treating with 5 u of RNase-free DNase-I (Roche Molecular Biochemicals)/10 μg of RNA. cDNA was synthesized using moloney leukemia virus reverse transcriptase with dNTPs and random hexamer primers (Stratagene). PCR synthesis for each primer pair was quantified at 15, 20, 25, and 30 cycles in a test reaction to ensure that the quantitative PCR amplification was in the linear range. For gene array analysis cDNA synthesis was performed with 20 μg of total RNA using the Superscript Choice cDNA Synthesis Kit (Invitrogen), employing an HPLC purified T7-Promoter-dT30 primer (Genset) to initiate the first-strand reaction. Biotin-labeled cRNA was synthesized from T7 cDNA using the RNA transcript labeling kit, Bio Array (Enzo), supplemented with biotin 11-CTP and biotin-16-UTP (Enzo) as specified by the Affymetrix technical manual. Biotin-labeled cRNA was fragmented in Tris (40 mM, pH 8.1), KOAc (100 mM), MgOAc (30 mM) for 30 min at 94° C. and hybridization samples were prepared according to the Affynmetrix manual. Genechip M430A and B probe arrays (Affymetrix) were hybridized, washed and stained according to the manufacturer's instructions in a fluidics station (Affymetrix). The arrays were scanned using a Hewlett Packard confocal laser scanner and visualized using Genechip 5.1 software (Affymetrix).

Laboratory Measurements

Blood samples were taken from mice using non-heparinized capillary tubes. Liver cytosolic samples were obtained by dounce homogenization in PBS buffer (0.005% Triton-X-100) and centrifugation. Insulin was quantified using a radioimmunoassay (NEN). Ketone bodies and free fatty acids were quantified using a colorimetric assay system (Wako Chemicals). Glucose was measured using a standard glucose sensor (Glucometer Elite, Bayer). Cholesterol and Triglycerides were determined using a colorimetric assay system (Roche).

Liver Perfusion

After anesthesia with pentobarbitone sodium (60 mg/kg i.p.), the portal vein and the inferior vena cava were cannulated. The liver was perfused with oxygenated Krebs-Henseleit buffer with varying amounts of glucose and insulin at 37° C. in a single-pass mode with a total flow rate of 1.5-2 ml/min.

Mitochondrial β-Oxidation

Mitochondria from perfused livers of mice were isolated by differential centrifugation as described by Hoppel et al. (1979) J. Biol. Chem. 254-4164-4170. An aliquot of freshly isolated mitochondria was used to determine mitochondrial protein. The β-oxidation of [1-¹⁴C]palmitic acid by liver mitochondria was assessed as described by Lang et al. (2001) J. Lipid Res. 42:22-20. CO₂ trapped on the filter papers was counted for 1-¹⁴C activity using a scintillation counter. The incubation mixture was centrifuged at 4,000 g for 10 min and an aliquot of the supernatant was counted for 1-¹⁴C activity. This activity measures acid-soluble products of mitochondrial palmitate metabolism, which equals the formation of ketone bodies. (Freneaux et al. (1988) Hepatology 8:1056-62).

Statistical Analysis

Results are given as mean±SD. Statistical analyses were performed by using a Student's t-test, and the null hypothesis was rejected at the 0.05 level. Linear regression was calculated using Origin (Microcal).

### EXAMPLE 14

### Nuclear Localization of Foxa-2 is Tightly Regulated by Circulating Insulin Levels

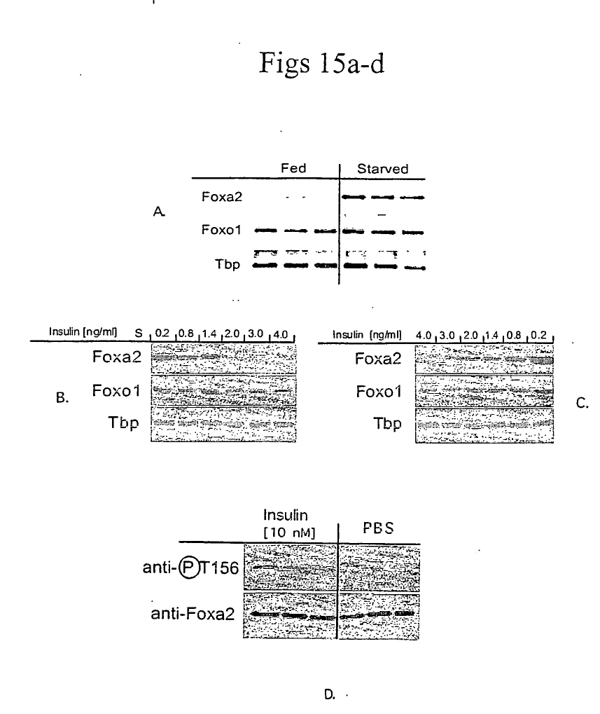

The role of Foxa-2 in the regulation of liver gene expression by nutritional status was examined by determining the intracellular localization of Foxa-2 in fasted and fed mice by Western blotting of Foxa-2 in liver nuclear extracts and by immunohistochemistry. In fed mice, nuclear Foxa-2 levels were low, whereas after an overnight fast Foxa-2 expression in liver nuclei increased ^{˜}4 to 5-fold (FIG. 15a). The nuclear localization of Foxo1, a related forkhead transcription factor, was only increased 2-fold in starved compared to the fed state. To test if the nuclear exclusion of Foxa-2 in the fed state was mediated by insulin and to explore the temporal relationship between insulin action and nuclear translocation, either PBS (control) or 20 ng/ml insulin was injected into the portal vein of fasted mice. Liver sections were fixed after 5 and 15 minutes and stained with anti-Foxa-2 antibodies and counterstained with TOPRO-3 dye to visualize nuclei. Strong Foxa-2 immunostaining was detected in hepatocyte nuclei of control (PBS) mice. In contrast, in livers injected with insulin, Foxa-2 was excluded from ^{˜}80% of nuclei after 5 minutes and located exclusively in the cytoplasm of all hepatocytes 15 minutes after insulin administration. Foxa-2 protein levels were measured in liver nuclei of C57/B6 mice that were perfused with a buffer containing a range of insulin concentrations that included fasting and postprandial insulin levels (0.2 and 4.0 ng/ml, respectively). Livers of fasted mice were perfused through the portal vein at 1-2 ml/min with buffer containing 0, 0.2, 0.8, 1.4, 2.0, 3.0 and 4.0 ng/ml insulin. Each perfusion was administered for 20 minutes and liver samples were obtained for the isolation of hepatocyte nuclei at the end of each period. Foxa-2 levels were determined by immunoblotting. An inverse relationship between insulin levels and concentrations of nuclear Foxa-2 was observed with barely detectable levels at 2.0 ng/ml (FIG. 15b). Similar data were obtained when livers were first perfused with high insulin concentrations (4 ng/ml) and insulin levels were gradually decreased (FIG. 15c). There was a >20-fold change in nuclear Foxa-2 expression between livers that were exposed to low (fasting) and high (postprandial) insulin levels. The regulation of nuclear/cytosol localization of Foxo1 by insulin was less pronounced (FIG. 15b, c). To confirm that amino acid residue TI56 in the Foxa-2 protein is the site that is phosphorylated in vivo, antibodies were raised to phosphopeptides that correspond to this phosphorylation site and the antibodies were used in immunoblotting experiments. Foxa-2 was immunoprecipitated from whole cell extracts of livers that received an intraportal injection of 20 ng/ml insulin or PBS. Immunoblots were then probed with antibodies recognizing specifically phosphorylated Foxa-2 (anti-TI56), or both phosphorylated and unphosphorylated, Foxa-2 (anti-Foxa-2) (FIG. 15d). The anti-phospho TI56 peptide antibody recognized phosphorylated Foxa-2 in livers treated with insulin but failed to detect Foxa-2 in hepatocytes that were not stimulated with insulin. Detection of Foxa-2 using anti-Foxa-2 antibodies showed that Foxa-2 protein levels were the same in treated and untreated livers. Together, these findings indicate that Foxa-2 activity in the liver is tightly regulated by circulating insulin levels through a mechanism that involves phosphorylation at position TI56, followed by nuclear exclusion and inhibition of gene expression.

### EXAMPLE 15

### Foxa-2 Activates Genes Involved in β-Oxidation and Ketogenesis

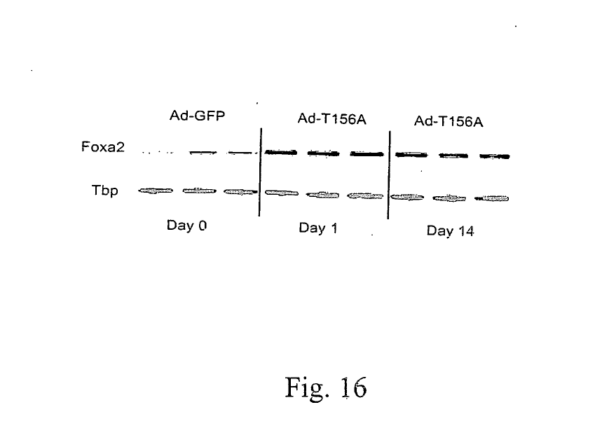

The foregoing examples demonstrate that activation of the insulin/PI3-kinase/Akt pathway induces Foxa-2 phosphorylation at a single conserved site (TI56). A mutant (TI56A) Foxa protein is resistant to Akt mediated phosphorylation, nuclear exclusion and transcriptional inactivation of Foxa-2-regulated genes in vitro. (Wolfrum et al. (2003) Proc. Natl. Acad. Sci. USA 100:11624-9). A recombinant adenovirus expressing Foxa-2TI56A (Ad-TI56A) was generated and used to test the expression and nuclear localization by immunohistochemistty in livers of mice following tail vein injections. Empty virus expressing only GFP (Ad-GFP) served as control. Ad libitum fed mice were infected with Ad-GFP or Ad-TI56A. Livers were fixed and stained with Anti-Foxa-2 antibodies and TOPRO-3 dye at 1 or 14 days after infection. Livers of fed mice showed strong nuclear staining of Foxa-2 at day 1 that persisted through day 14. In contrast, mice infected with Ad-GFP only revealed immunostaining in the cytosol of hepatocytes. Western blot analysis of nuclear Foxa-2 in livers of 6 hr fasted animals infected with Ad-GFP or Ad-TI56A showed that Foxa-2 was respectively 3 and 2-fold increased at 1 and 14 days post infection, compared to Ad-GFP infected mice (FIG. 16). These data indicate that infection with Ad-TI56A leads to constitutive nuclear expression of Foxa-2 in the liver. Furthermore, it shows that the increase in nuclear Foxa-2 expression after adenoviral infection is similar than the observed rise in nuclear expression in livers of fasted mice.

Gene expression profiles were generated from livers of mice that were infected with either Ad-GFP or Ad-TI56A using Aftmetrix™ oligonucleotide expression arrays. Gene expression was measured in randomly fed animals at day 1 and day 14 post infection to capture acute and secondary changes in transcriptional profiles. Several clusters of genes involved in lipid and fatty acid metabolism were identified that were upregulated in livers infected with Foxa-2TI56A compared to control virus (Table 2). These included lipases (hepatic lipase, lipoprotein lipase, endothelial lipase, monoglyceride lipase), genes involved in the transport of fatty acids into cells (Cd36) as well as mitochondria (enzymes of carnitine metabolism, carnitine acyltransferase 1, carnitine translocase, fatty acid CoA ligase) and several genes encoding enzymes of mitochondrial and peroxisomal β-oxidation (Table 2). In addition, MRNA levels of key enzymes of ketogenesis, acetyl CoA ligase, HMG CoA synthase and 3-hydroxybutyrate dehydrogenase, were increased in livers expressing the constitutively active Foxa-2 protein. The analysis also revealed an increase in expression of several key enzymes of carbohydrate metabolism, including glucok-inase, pyruvate kinase and glucose 6 phosphatase (G6pc) (Table 2). It was also found that Ad-TI56A expressing livers had increased expression of other important genes in glucose and lipid metabolism, including transcription factors (Foxal, Foxa3, Hnf4α, Pparγ), uncoupling proteins Ucp2 and Ucp3, and insulin degrading enzyme Ide. Together, these changes in hepatic gene expression indicate that Foxa-2 is a regulator of fatty acid metabolism and ketogenesis and also has a role in glucose metabolism and insulin sensitization of hepatocytes. Table 2 shows fold regulation of gene expression measured 24 hours after adenoviral infection.

TABLE 2

| Gene name | Fold-regulation |
|---|---|
| Triglyceride degradation | |
| Lipoprotein lipase | 2.0 |
| Hepatic lipase | 2.5 |
| Endothelial lipase | 6.7 |
| Monoglyceride lipase | 2.5 |
| Glyerol kinase | 3.0 |
| Hormone sensitive lipase | 2.0 |
| Mitochondrial fatty acid import | |
| CD36 | 3.3 |
| Butyrobetaine (gamma), 2-oxoglutarate dioxygenase 1 | 2.5 |
| Carnitine acetyltransferase | 3.7 |
| Carnitine octanoyltransferase | 2.0 |
| Carnitine palmitoyltransferase 1, liver | 2.7 |
| Carnitine translocase (Slc25a20) | 2.2 |
| Fatty acid Coenzyme A ligase, long chain 2 | 3.0 |
| Mitochondrial β-oxidation (saturated & unsaturated FA | |
| Acetyl-CoA DH very long chain | 2.5 |
| Acetyl-CoA DH medium chain | 2.7 |
| Mitochondrial acyl-CoA thioesterase | 6.7 |
| Hydroxyacyl-CoA dehydrogenase type II | 2.2 |
| 3-Ketoacyl-CoA thiolase B | 2.2 |
| Enoyl-CoA isomerase | 2.5 |
| Peroxisomal β-oxidation | |
| Peroxisomal biogenesis factor 11a | 2.0 |
| Enoyl-CoA hydratase 1 | 1.8 |
| Peroxisomal acyl-CoA thioesterase 2B | 4.5 |
| Acyl-CoA oxidase 1 | 1.8 |
| 2-4-dienoyl-CoA reductase 2 | 1.8 |
| Ketone body formation | |
| Acetoacetyl-CoA Synthase | 2.2 |
| 3-Hydroxybutyrate dehydrogenase | 3.0 |
| HMG CoA synthase | 3.7 |
| Fatty acid synthesis | |
| Acetyl-CoA carboxylase | −2.7 |
| Fatty acid synthase | −2.0 |
| Steroyl-CoA dehydrogenase | −3.0 |
| Glucose metabolism | |
| Glucokinase | 3.7 |
| L-Pyruvate kinase | 1.8 |
| M-Pyruvate kinase | 2.5 |
| Glucose 6 phosphatase (catalytic subunit) | 5.0 |
| Transcription factors | |
| Foxa1 | 2.5 |
| Foxa-2 | 2.5 |
| Foxa3 | 3.3 |
| Hepatocyte nuclear factor 4a (Hnf4a) | 2.0 |
| Pparγ | 2.7 |
| Other | |
| Ucp2 | 2.7 |
| Ucp3 | 3.7 |
| L-Fabp | 2.7 |
| Insulin degrading enzyme | 2.0 |

To examine the physiological consequences of Foxa-2 mediated gene activation, oxidative metabolism of palmitate by isolated liver mitochondria of mice infected with Ad-TI56A or control Ad-GFP virus was assayed. Mitochondria were incubated in the presence of [1-¹⁴C]palmitic acid and the formation of ¹⁴C-ketone bodies and ¹⁴CO₂ from palmitate was determined. The generation of acid-soluble products (representing ketone bodies) was increased 2.4-fold in mitochondria of livers expressing Foxa-2TI56A compared to control virus (2.95±0.6 vs. 1.23±0.2 mmol/mg/min, p=0.006). This finding is consistent with increased ketogenesis in Foxa-2TI56A expressing hepatocytes, most likely due to increased activity of the HMG-CoA pathway. In addition, production of ¹⁴CO₂ from palmitate, determined in the same incubations as the formation of ketone bodies and reflecting the activity of β-oxidation, was also increased from mitochondria of livers infected with Ad-TI56A (0.082±0.01 vs 0.039±0.01 fmol/mg/min, p=0.009). Together, these data show that Foxa-2 is a transcriptional activator of fatty acid oxidative metabolism and ketogenesis.

### EXAMPLE 16

### Foxa-2TI56A Decreases Hepatic Triglyceride Content, Improves Hepatic Insulin Sensitivity and Normalizes Plasma Glucose Levels in Obese/Diabetic Mouse Models

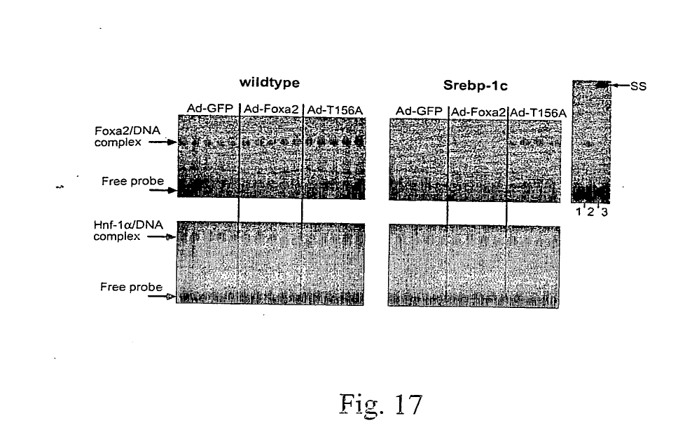

The nuclear exclusion of forkhead transcription factor Foxo-1 in response to insulin signaling has been shown to be impaired in animal models of obesity and insulin resistance, leading to a permanent nuclear localization, in spite of high circulating insulin levels. This observation has been suggested to contribute to overexpression of genes encoding key enzymes of gluconeogenesis and increased glucose production by the liver. (Altomonte et al. (2003) Am. J. Physiol. Endocrinol. Metab. 285:E718-28; Puigserver et al. (2003) Nature 500-555). The intracellular localization of Foxa-2 was examined in hepatocytes of three insulin resistant mouse models: the leptin deficient ob/ob, the lipoatrophic aP2-Srebp-1c, which expresses a transgene encoding a truncated dominant-positive fragment of SREBP-1c (amino acids 1-436) under control of the fat-specific aP2 promoter/enhancer, and high fat diet-induced obeseC57/B6 mice. Foxa-2 was localized exclusively in the cytosol of hepatocytes from starved and ad libitumn fed mice. These results indicate that Foxa-2 was permanently inactivated in hyperinsulinemic, insulin resistant mice. To examine the role of Foxa-2 activation in the pathogenesis of diabetes recombinant adenoviruses expressing GFP (Ad-GFP), wildtype Foxa-2 (Ad-Foxa-2) or constitutive active Foxa-2 (Ad-TI56A) were injected into wildtype, ob/ob, srebp-1c, and high-fat diet induced obese mice that had received a high fat diet for 12 weeks. Mice were sacrificed two weeks after adenoviral infection. Immunohistochemistry of recombinant Foxa-2 using anti-HA antibodies revealed that Foxa-2 was mainly localized in the cytoplasm, whilst Foxa-2TI56A was located in the nucleus. These results were confirmed by electrophoretic mobility shift assay (EMSA) analysis, using ³²P-labeled oligonucleotides that contained a Foxa-consensus binding site and liver nuclear extracts of mice injected with recombinant adenovirus. Specifically, DNA binding activity was measured using a ³²P-labeled double-stranded oligonucleotide containing a consensus Foxa binding site of the Igfbp as a probe. The supershift was performed with a monospecific anti-Foxa-2 antiserum (FIG. 17 top, right panel). EMSA analysis was also carried out with the same nuclear extracts and a labeled oligonucleotide probe containing an HNFI binding site (FIG. 17, lower panel). FIG. 17 shows that DNA/Foxa-2 complexes could be detected in nuclear extracts of livers of starved wildtype mice infected with control (Ad-GFP) or Ad-Foxa-2 but was absent or barely detectable in livers of starved ob/ob or Srebp-1c mice. In contrast, Foxa-2 DNA binding activity was identified an all livers of mice infected with Ad-TI56A. These data demonstrated that wildtype Foxa-2 expression by Ad-Foxa-2 was normally regulated and excluded from the nucleus in the postprandial state whilst Foxa-2TI56A was persistently located in the nucleus, irrespective of nutritional status.

The effect of constitutive Foxa-2 activation in livers of insulin resistant/diabetic mice on glucose and lipid metabolism was examined. Wildtype, ob/ob, Srebp-1c and high fat diet-induced obese mice were injected with Ad-GFP, Ad-Foxa-2 or Ad-TI56A and plasma glucose, insulin, triglyceride, free fatty acid and ketone body levels were assayed every 3 days over a period of two weeks. In addition, an insulin tolerance test was performed after two weeks of treatment. All measurements were performed after a moderate, 6 hour fasting period. No significant changes in plasma glucose levels were observed in C57/B6 mice injected with Ad-GFP, Ad-Foxa-2 or Ad-TI56A. However, plasma glucose concentrations profoundly decreased and essentially normalized in ob/ob, Srebp-1c and diet-induced obese mice that received Ad-Foxa-2TI56A injections. The decrease in glucose was accompanied by a significant fall in plasma insulin concentrations. In contrast no changes in metabolic end points were measured in animals infected with Ad-Foxa-2 compared to control Ad-GFP, supporting biochemical data that adenoviral expression of wildtype Foxa-2 is regulated by insulin and hence inactivated in obese mice. The insulin tolerance test revealed that insulin sensitivity markedly improved in diabetic mice treated with Ad-TI56A and compared to Ad-Foxa-2 and Ad-GFP injected animals. Plasma concentrations of triglycerides, free fatty acids and ketone bodies markedly increased during the 2-week treatment period in animals injected with Ad-TI56A compared to Ad-GFP infected mice, whilst liver triglyceride content decreased in Ad-T156A infected animals. Since expression of Foxa-2TI56A in obese mice elicited such profound changes in blood metabolites and hormonal status, further studies were performed to determine whether measurable outcomes of whole body metabolism were altered in Ad-TI56A treated animals. Weight, O₂-consumption, CO₂- and heat production were measured in ob/ob and Srebp-1c mice that were treated with Ad-GFP, Ad-Foxa-2 and Ad-TI56A. Obese mice exhibited a moderate weight loss of 3-4 grams after 14 days of treatment with Ad-TI56A compared to Ad-GFP treated animals (weight values). Resting oxygen consumption and CO₂-production were also significantly increased in mice expressing Foxa-2TI56A compared to Ad-Foxa-2 and Ad-GFP mice (O₂ consumption: ob/ob, 1712±36 vs. 1379±117 l/kg·h, P<0.01, Srebp-1c: 3901±489 vs. 3239±163 l/kg·h, P<0.05; CO₂ production: ob/ob, 1738±49 vs. 1356±81 l/kg·h, P<0.01, Srebp-1c, 3765±397 vs. 3174±269 l/kg·h, P<0.05). Furthermore, heat production of mice under resting conditions was also increased in ob/ob mice (533±28 vs. 424±11 kcal/h, P<0.01). Taken together, these data indicate that active Foxa-2 is a powerful regulator of glucose homeostasis, lipid metabolism and insulin sensitivity in obese mouse models of diabetes.

### EXAMPLE 17

### Foxa-2 Improves Hepatic Insulin Senisitivity in Livers of Obese Mice

To study the transcriptional profiles that may be altered by nuclear localization of Foxa-2 in obese mice, gene expression was compared in ob/ob and srebp-1c livers that were infected with Ad-GFP and Ad-TI56A. Semiquantitative RT-PCR analysis was performed to assay the expression of representative genes in the liver of mice at 14 days postinfections. Fold-change was calculated after densitometr), of amplified products and determination of ratios of expression levels of Ad-GFP and Ad-TI56A infected livers. Measurements were obtained from 4-5 mice in each group two weeks post infection. At that time, Foxa-2 mRNA and protein levels of recombinant Foxa-2TI56A were only two-fold increased in Ad-TI56A treated animals compared to mice treated with control virus (FIG. 16). Similar to gene expression profiles in wildtype mice, robust increases were observed in expression levels of genes involved in triglyceride degradation, mitochondrial fatty acid transport, mitochondria and peroxisomal β-oxidation, ketogenesis, and glycolysis (Table 3). In addition, the expression of hepatic lipase, peroxisomal proliferating activator gamma and uncoupling proteins 2 and 3 (Ucp2/3) were increased in Ad-TI56A infected mice, while FAS and SCD-1, two key regulatory enzymes involved in fatty acid synthesis, were decreased.

TABLE 3

| | Srep-1c | ob/ob | HF |
|---|---|---|---|
| Triglyceride degradation | | | |
| Hormone sensitive lipase | 1.6 | 2.2 | 2.0 |
| Endothelial Lipase | 3.1 | 2.8 | 3.0 |
| Hepatic Lipase | 2.7 | 3 | 2.3 |
| Glycerol Kinase | 2.4 | 2.3 | 2.3 |
| Mitochondrial fatty acid import | | | |
| CD36 | 4.2 | 3.1 | 2.8 |
| Fatty acyl-CoA Ligase II | 1.5 | 1.6 | 1.6 |
| CPT | 3.9 | 4.2 | 3.9 |
| Mitochondrial β-oxidation | | | |
| Acetyl-CoA dehydrogenase very long chain | 1.4 | 1.9 | 2.3 |
| Acetyl-CoA dehydrogenase medium chain | 2.5 | 2.2 | 2.2 |
| Hydroxyacyl-CoA dehydrogenase | 0.9 | 0.9 | 1.0 |
| 3-Ketoacyl-thiolase | 2.1 | 2.3 | 2.1 |
| 2,4-dienoyl-CoA reductase | 2.3 | 2.2 | 2.1 |
| Peroxisomal β-oxidation | | | |
| Acyl-CoA oxidase | 2.3 | 2.4 | 2.3 |
| Ketone body formation | | | |
| Acetoacetyl-CoA Synthase | 1.7 | 1.9 | 1.9 |
| HMG-CoA Synthetase | 1.8 | 2 | 2.5 |
| Hydroxybutyrate dehydrogenase | 1.8 | 2.1 | 2.1 |
| Fatty acid synthesis | | | |
| Fatty acid synthase | −1.9 | −1.8 | −1.8 |
| stearoyl-CoA dehdrogenase | −2.1 | −2.2 | −2.0 |
| Transcription factors | | | |
| Foxal | 1.2 | 1.2 | 1.2 |
| Foxa-2 | 1.8 | 1.8 | 2.1 |
| HNF4α | 1.6 | 1.6 | 1.5 |
| PPARγ | 1.7 | 1.7 | 2.1 |
| Glucose Metabolism | | | |
| Glucokinase | 2.5 | 2.6 | 2.1 |
| Glucose-6-Phoshatase | 1.3 | 1.4 | 1.3 |
| Pyruvate Kinase, liver | 1.3 | 1.5 | 1.4 |
| Pyruvate Kinase, muscle | 1.7 | 1.8 | 2.1 |
| Other | | | |
| UCP2 | 1.9 | 1.9 | 1.8 |
| UCP3 | 3.6 | 3.1 | 2.4 |
| L-FABP | 1.7 | 2 | 1.8 |

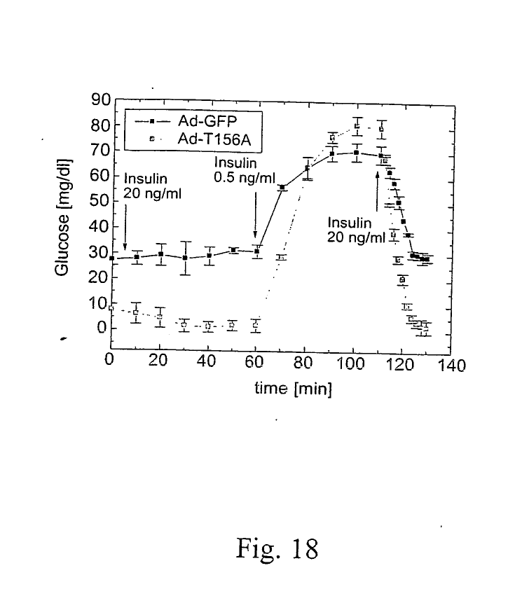

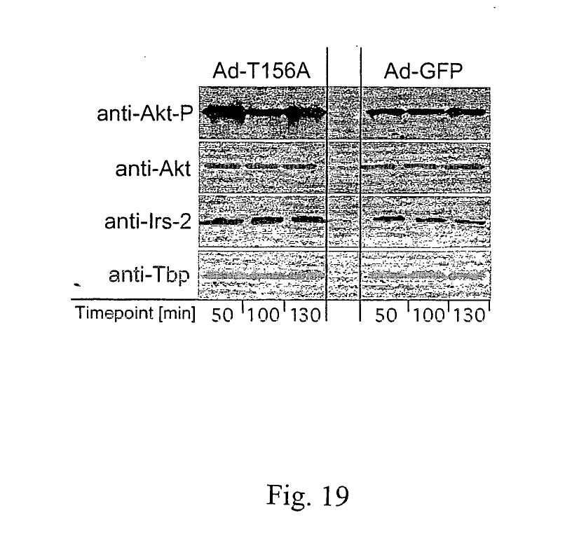

The physiological effects of Ad-TI56A expression on hepatic insulin sensitivity and glucose production were examined. Isolated livers of Ad-GFP or Ad-T156A infected ob/ob mice were perfused with 20 ng/ml of insulin for one hour and glucose concentrations were measured in the effluate during this period. Livers expressing Foxa-2TI56A had an approximately 3-fold reduction in glucose output compared to control livers (FIG. 18). Glucose output from the liver increased significantly when insulin concentrations were decreased to 0.5 ng/ml. Under these conditions, glucose output of ob/ob livers infected with Ad-TI56A exceeded the glucose production of livers that were infected with control Ad-GFP virus. Liver extracts of ob/ob livers infected with Ad-GFP or Ad-TI56A were also prepared and the levels of IRS-2 and total and phosphorylated Akt after 50 min of each low (0.5 ng/ml) and high (20 ng/ml) insulin perfusion were determined. Levels of IRS-2 and phosphorylated Akt were increased more than 2-fold in livers of Ad-TI56A compared to Ad-GFP treated animals, while total Akt levels remained constant, indicating that insulin signaling was improved in livers expressing Foxa-2_{T156A}. Results are shown in FIG. 19. These results indicate that Foxa-2TI56A increased hepatic insulin sensitivity and markedly diminished glucose output in livers of ob/ob animals.

### EXAMPLE 18

### Decreased Fatty Acid Metabolism and Hepatic Insulin Sensitivity in Foxa-2^{+/-} Animals

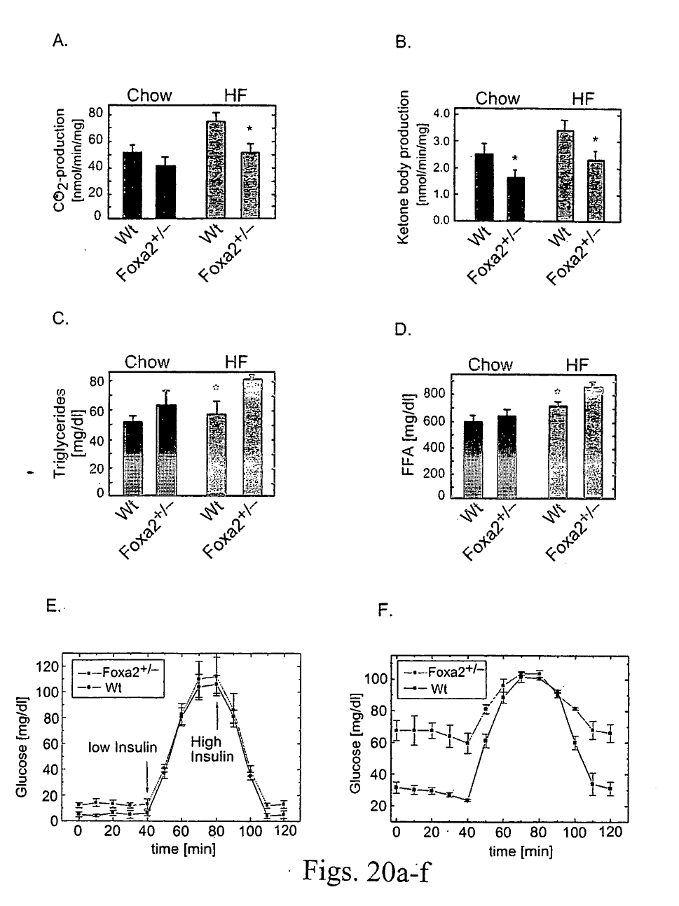

Examples 15-17 utilized a phosphorylation-deficient, constitutive active form of Foxa-2 that does not leave the nucleus and therefore leads to the forced expression of Foxa-2 target genes. The following example was performed to determine whether diminished expression of hepatic Foxa-2 whose activity can still be regulated by circulating insulin levels would have an effect on glucose metabolism and lipid oxidation. Mutant Foxa-2 mice that have one inactivated Foxa-2 allele (Foxa-2^{+/-}) by targeted insertion of the LacZ gene were used. (Weinstein et al. (1994) Cell 78:575-588). These haploinsufficient Foxa-2 mice were used because Foxa-2 null mice have an early embryonic lethal phenotype (at ^{˜}E7.5) and heterozygous mice exhibit no overt metabolic phenotype. (Weinstein et al. (1994) Cell 78:575-588). Foxa-2^{+/-} mice and wildtype littermates were fed either a chow or a high fat (55% fat) diet. Interestingly, fasted Foxa-2^{+/-} mice had increased plasma free fatty acid concentration compared to control littermates after a 12 week high fat diet (836±44 vs. 728±36 μM, P<0.05, n=8). To examine lipid oxidation in these animals, mitochondria were isolated from livers perfused with fasting levels (0.5 ng/ml) of insulin. The formation of CO₂ and ketone bodies with [¹⁴C] palmitate as a substrate was compared between wildtype and Foxa-2^{+/-} mice. Livers of Foxa-2^{+/-} animals on a chow diet exhibited significantly decreased k-etogenesis and borderline reduction in β-oxidation. Fatty acid metabolism was increased in livers of animals on a high fat diet compared to chow fed mice but Foxa-2^{+/-} livers had diminished CO₂ and ketone body production, reflecting the lower activity of β-oxidation and reduced activity of the HMG-CoA pathway (FIG. 20). These results are also consistent with increased liver triglyceride content in livers and higher plasma triglyceride and free fatty acid levels in diet-induced obese Foxa-2^{+/-} mice compared to wildtype littermates (Example 16). Hepatic insulin sensitivity in high fat diet induced Foxa-2^{+/-} and wildtype littermates was also determined. No significant difference in glucose output was observed in perfused livers of chow fed mice; however, Foxa-2^{+/-} animals on a high fat diet had an average glucose output of >200% compared to wildtype littermates on the same diet. Together, these data show that reduced expression of Foxa-2 is normally sufficient to maintain normal glucose and lipid homeostasis but leads to defects in fatty acid oxidation and increased hepatic insulin resistance under conditions of high caloric feeding.

The foregoing examples demonstrate that circulating insulin levels regulate the activity of Foxa-2 by nuclear/cytosolic localization and that Foxa-2 is an activator of the β-oxidation pathway and ketogenesis. In ad libitum fed mice, Foxa-2 is mostly located in the cytosol and translocates into the nucleus during starvation. The exclusion of Foxa-2 from the nucleus is mediated by phosphorylation of an evolutionarily conserved tyrosine residue at position 156 in response to circulating insulin. This was demonstrated by a specific phosphopeptide antibody, which recognized phospho-TI56 of the Foxa-2 protein in whole cell extracts of livers treated with insulin, but which failed to detect Foxa-2 in liver extracts of starved mice. The tight regulation of Foxa-2 by insulin was further demonstrated in livers perfused with increasing or decreasing concentrations of insulin, demonstrating an inverse dose-response relationship between insulin levels and nuclear Foxa-2 localization. The process of nuclear translocation of Foxa-2 in response to insulin stimulation is fast and leads to an >20-fold increase in nuclear Foxa-2 levels within 5-20 minutes. Glucagon does not affect Foxa-2 nuclear localization in liver perfusion experiments, demonstrating that the activation of Foxa-2 activity by nuclear translocation during starvation is due to the lack of insulin signaling and independent of glucagon/cAMP activation.

The greatest change of gene expression in an entire pathway was detected in triglyceride/fatty, acid metabolism. Foxa-2 activated expression of lipases, membrane associated fatty acid transporters, most enzymes for mitochondrial β-oxidation of long and medium chain fatty acids including the rate-limiting steps and their transport across the mitochondrial membrane via the carnitine shuttle system. Genes responsible for peroxisomal β-oxidation were also increased in expression. Importantly, genes encoding enzymes that synthesize and release ketone bodies were robustly upregulated in livers infected with constitutive active Foxa-2. In contrast, decreased expression of fatty acid synthase (FAS) and steroyl-CoA desaturase (SCD-1), which are key enzymes for fatty acid synthesis, was found. The physiological significance of activation in gene expression in fatty acid oxidation and ketogenesis pathways was confirmed by the increase in β-oxidation and ketone body formation using mitochondria of livers from mice infected with Ad-TI56A and compared to Ad-GFP treated animals. In conjunction with the results demonstrating that β-oxidation and ketone body production is decreased in Foxa-2^{+/-} mice when fed a high fat diet, these data demonstrate that Foxa-2 is a sensor of low circulating insulin levels that mediates the metabolic adaptation to starvation of liver by inducing the expression of gene clusters involved in lipid catabolism. Foxa-2^{+/-} mice on a regular chow diet exhibited no alteration of lipid β-oxidation or ketone body production. Thus, Foxa-2 is required under challenging conditions like high caloric intake or prolonged starvation.

Livers expressing Foxa2TI56A had elevated levels of steady state phospho-Akt compared to control animals. Furthermore, insulin markedly induced expression of Irs-2 and phosphorylation of Akt in Ad-TI56A treated animals, whilst Akt phosphorylation could not be further stimulated by insulin in Ad-GFP infected ob/ob mice. The profound improvement of hepatic insulin resistance by Foxa-2 was further demonstrated by the marked reduction in liver glucose output under hyperinsulinemic conditions in obese mice infected with Ad-TI56A. Conversely, reduced Foxa-2 expression in Foxa-2^{+/-} mice when challenged by a high fat diet, led to increased insulin resistance and increased hepatic glucose output. The increase in insulin sensitivity may also be influenced by the breakdown of liver triglycerides and stimulation of fatty acid oxidation in Ad-TI56A treated mice, thereby improving the hepatic steatosis of these animals, while Foxa-2^{+/-} mice on a high fat diet accumulate more liver triglycerides. The expression of two rate limiting glycolyrtic enzymes, glucokinase and pyruvate kinase also increased, indicating that Foxa-2 also promotes carbohydrate metabolism.

1. A method for identifying an agent that increases Foxa-2 expression comprising contacting a plurality of cells that contain a Foxa-2 promoter operably linked to a coding sequence for Foxa-2 or a reporter gene with a candidate agent; assaying for expression of Foxa-2 or the reporter in the presence and absence of the candidate agent; and comparing Foxa-2 or reporter expression in the presence and absence of the candidate agent, whereby an increase in Foxa-2 or reporter expression in the presence of the candidate agent is indicative of the identification of an agent that increases Foxa-2 expression.

2. The method of claim 1 wherein the cells are mammalian cells.

3. The method of claim 2 wherein the cells are human cells.

4. The method of claim 3 wherein the cells are human preadipocytes or adipocytes.

5. The method of claim 1 wherein the cells are 3T3-L1 cells.

6. The method of claim 1 wherein the cell contains a construct comprising a Foxa-2 promoter operably linked to a coding sequence for Foxa-2.

7. The method of claim 6 wherein the coding sequence encodes an adipocyte-specific Foxa-2 isoform.

8. The method of claim 1 wherein Foxa-2 expression is assayed by detecting Foxa-2 mRNA.

9. The method of claim 8 wherein Foxa-2 mRNA is detected by Northern blotting or polymerase chain reaction.

10. The method of claim 1 wherein Foxa-2 expression is assayed by detecting Foxa-2 protein.

11. The method of claim 10 wherein Foxa-2 protein is detected by Western blotting or immunohistochemistry.

12. The method of claim 1 wherein the reporter gene is selected from the group consisting of the chloramphenicol acetyl transferase gene, the beta-galactosidase gene, the beta-glucuronidase gene, the green fluorescence protein gene and the luciferase gene.

13. The method of claim 1 wherein the cell is 3T3-L1 cell stably transformed with a construct comprising a Foxa-2 promoter operably linked to the coding sequence of the luciferase gene.

14. A composition comprising an agent identified by the method of claim 1.

15. A method for identifying an agent that increases Fxr expression comprising contacting a plurality of cells that contain a Fxr promoter operably linked to a coding sequence for Fxr or a reporter gene with a candidate agent; assaying for expression of Fxr or the reporter in the presence and absence of the candidate agent; and comparing Fxr or reporter expression in the presence and absence of the candidate agent, whereby an increase in Fxr or reporter expression in the presence of the candidate agent is indicative of the identification of an agent that increases Fxr expression.

16. The method of claim 15 wherein the cells are mammalian cells.

17. The method of claim 16 wherein the cells are human cells.

18. The method of claim 17 wherein the cells are human preadipocytes or adipocytes.

19. The method of claim 15 wherein the cells are 3T3-L1 cells.

20. The method of claim 15 wherein the cell contains a construct comprising a Fxr promoter operably linked to a coding sequence for Fxr.

21. The method of claim 20 wherein the coding sequence encodes human Fxr.

22. The method of claim 15 wherein Fxr expression is assayed by detecting Fxr mRNA.

23. The method of claim 22 wherein Fxr mRNA is detected by Northern blotting or polymerase chain reaction.

24. The method of claim 15 wherein Fxr expression is assayed by detecting Fxr protein.

25. The method of claim 24 wherein Fxr protein is detected by Western blotting or immunohistochemistry.

26. The method of claim 15 wherein the reporter gene is selected from the group consisting of the chloramphenicol acetyl transferase gene, the beta-galactosidase gene, the beta-glucuronidase gene, the green fluorescence protein gene and the luciferase gene.

27. The method of claim 15 wherein the cell is 3T3-L1 cell stably transformed with a construct comprising a Fxr promoter operably linked to the coding sequence of the luciferase gene.

28. A composition comprising an agent identified by the method of claim 15.

29. A method of identifying an agent that activates Fxr comprising contacting a plurality of cells that contain Fxr with a candidate agent; assaying for activation of Fxr in the presence and absence of the candidate agent; and comparing activation of Fxr in the presence and absence of the candidate agent, wherein an increase in activation in the presence of the agent is indicative of the identification of an agent that activates Fxr.

30. The method of claim 29 wherein the cells contain a vector comprising an Fxr promoter operably linked to a reporter gene, and activation of Fxr is assayed by measuring reporter gene activity.

31. The method of claim 29 wherein Fxr activation is assayed by measuring increased expression of Fxr target genes.

32. A composition comprising an agent identified by the method of claim 29.

33. A method of inhibiting adipogenesis comprising contacting a cell with an agent identified by the method of any one of claims 2, 15 and 29.

34. A method for treating obesity, metabolic syndrome or Type 2 diabetes comprising administering to a subject in need of such treatment a composition comprising an agent identified by the method of any one of claims 1, 15 and 29.

35. A method for inhibiting adipogenesis comprising contacting a cell capable of adipogenesis with an agent selected from the group consisting of an agent that increases levels of Foxa-2 mRNA, an agent that increases levels of Foxa-2 protein, an agent that increases levels of Fxr mRNA, an agent that increases levels of Fxr protein, and an agent that activates Fxr.

36. The method of claim 35 wherein the agent that increases levels of Foxa-2 protein is Foxa-2 protein or a vector that expresses Foxa-2 protein.

37. The method of claim 35 wherein the agent that increases levels of Fxr protein is Fxr protein or a vector that expresses Fxr protein.

38. A method for treating obesity, metabolic syndrome or Type 2 diabetes comprising administering to a subject in need of such treatment a composition comprising an agent selected from the group consisting of an agent that increases levels of Foxa-2 mRNA, an agent that increases levels of Foxa-2 protein, an agent that increases levels of Fxr mRNA, an agent that increases levels of Fxr protein, and an agent that activates Fxr.

39. A method of identifying an agent that inhibits the phosphorylation of Foxa-2 comprising combining a candidate agent with a polypeptide having Akt kinase activity and a substrate comprising the phosphorylation domain of Foxa-2; assaying for phosphorylation of the substrate in the presence and absence of the candidate agent; and comparing phosphorylation in the presence and absence of the candidate agent, whereby a decrease in phosphorylation of the substrate in the presence of the candidate agent is indicative of the identification of an agent that inhibits phosphorylation of Foxa-2.

40. The method of claim 39 wherein the polypeptide having Akt kinase activity is human Akt 1 or human Akt 2.

41. The method of claim 39 wherein the substrate is a Foxa-2 protein or fragment thereof comprising the phosphorylation domain.

42. The method of claim 39 wherein the substrate is human Foxa-2.

43. A composition comprising an agent identified by the method of claim 39.

44. A method of identifying an agent that inhibits the nuclear exclusion of Foxa-2 in hepatocytes comprising contacting a plurality of hepatocytes, under conditions whereby Foxa-2 exhibits nuclear exclusion, with a candidate agent; determining the intracellular location of Foxa-2 in the presence and absence of the candidate agent; and comparing the intracellular location of Foxa-2 in the presence and absence of the agent, whereby an increase in nuclear localization of Foxa-2 in the presence of the candidate agent is indicative of the identification of an agent that inhibits nuclear exclusion of Foxa-2 in hepatocytes.

45. The method of claim 44 wherein the hepatocrtes are HepG2 cells.

46. The method of claim 44 wherein the hepatocytes are contained within the liver of a mammal.

47. The method of claim 44 wherein intracellular location of Foxa-2 is determined by Western blotting, immunohistochemistry, or measurement of expression of Foxa-2-activated genes.

48. A composition comprising an agent identified by the method of claim 44.

49. A method of treating obesity, type 2 diabetes or hyperinsulinemia comprising administering to a subject in need of such treatment the composition of claim 39.

50. A method of treating obesity, type 2 diabetes or hyperinsulinemia comprising administering to a patient in need of such treatment the composition of claim 48.
